(19) United States
(12) Patent Application Publication
(10) Pub. No.: US 2026/0026923 A1
(43) Pub. Date: Jan. 29, 2026

(54) METHOD AND SYSTEM FOR MICRO NEAR INFRARED DETECTION OF AMINO ACID LEVELS IN BREAST MUSCLE

(71) Applicant: Tyson Foods, Inc., Springdale, AR (US)

(72) Inventors: Vivian Breen, Springdale, AR (US); Valerie Gunsaulis, Springdale, AR (US); William Herring, Springdale, AR (US); Fernando Brito Lopes, Springdale, AR (US); Marcos Yamaki, Springdale, AR (US)

(21) Appl. No.: 19/277,200

(22) Filed: Jul. 22, 2025

Related U.S. Application Data
(60) Provisional application No. 63/674,708, filed on Jul. 23, 2024.

(51) Int. Cl. A61D 19/00; G16B 40/10; G16B 40/20

(52) U.S. Cl. CPC A61D 19/00; G16B 40/10; G16B 40/20

(57) ABSTRACT

The technology as disclosed herein includes systems, methods, and compositions for detection of disease in a subject using spectroscopy and includes more effective detection of muscular myopathies in commercially farmed animals using infrared spectroscopy, and includes detection of Wooden Breast, White Striping, and/or Spaghetti Meat in broiler chickens utilizing near infrared spectroscopy. Implementations of the systems, methods, and compositions provided herein include and/or utilize and/or provide for detection of Wooden Breast, White Striping, and/or Spaghetti Meat in broiler chickens utilizing near infrared spectroscopy of breast area of live birds for prediction of taurine levels in the muscle.

### CROSS-REFERENCE TO RELATED APPLICATIONS

This Non-Provisional Utility Patent Application entitled Method and System For Micro Near Infrared Detection Of Amino Acid levels claims priority to and the benefit of Provisional Patent Application Ser. No. 63/674,708 filed Jul. 23, 2024, entitled NEAR INFRARED DETECTION OF DISEASE, which is incorporated herein by reference in its entirety.

### FEDERALLY SPONSORED RESEARCH OR DEVELOPMENT

N/A

### FIELD OF THE INVENTION

The present invention relates generally to systems, methodologies, processes, and compositions for detection of disease in a subject using spectroscopy, and more particularly to the detection of Wooden Breast, White Striping, and/or Spaghetti Meat in broiler chickens utilizing near infrared spectroscopy.

### BACKGROUND

The industrial poultry farming community has been challenged with an increased occurrence of several breast myopathies in recent years. These abnormalities often affect the pectoralis major muscle of fast-growing poultry products that are often selected for their production performances in areas such as: high growth rate, breast yield, and total weight. Examples of these myopathies are often referred to as White Striping, Wooden Breast (Woody Breast), and Spaghetti Meat. These diseases are impacting the muscle health and meat quality of poultry, and more particularly commercial broiler chickens and their economic returns.

Wooden Breast, for example, was described in 2014 and is characterized by the severe hardening of the pectoralis major muscle during growth to market weight for commercial chickens. The condition causes the breast muscle to become unusually hard and rubbery, often with a pale and bulging appearance. This altered texture and appearance make the meat less desirable to consumers, leading to a reduction in its marketability. In severe cases, the meat may be downgraded or even discarded, resulting in direct financial losses for producers. The economic impact is further compounded by the high prevalence of the condition in some flocks, where up to half of the birds may be affected. The actual rate of Wooden Breast disease in commercial settings is often not well documented for various industrial and practical reasons. However, Wooden Breast disease has been identified as an emerging quality defect. The affected poultry flocks have been reported at rates of up to 50% occurrence. Processes and systems are needed to not only develop strains or flocks through selection where Wooden Breast is less prevalent, but also systems and processes needed in a production environment to predict Wooden Breast for selective removal from the production line.

Spatiotemporal distribution has been shown for these disorders; the early stage often hits the superficial area in the cranial portion of the muscle, and then over time can include the entire muscle area. These myopathies all exhibit distinctive phenotypes however they often share common histological features, and thus it has been speculated that there may be common causative mechanisms responsible for the physiological and structural perturbations in the muscle associated with these conditions and might underpin their occurrence. For example, sarcoplasmic reticulum stress, principally induced by accumulated proteins which are misfolded may be responsible for the onset of these growth-related myopathies. But this is also driven by other factors such are altered calcium homeostasis and accumulation of fatty acids.

These diseases are impacting multiple industrial farming communities around the world, for example in the United States, Brazil, Italy, and Finland; and they have the prospect of affecting other world sectors as production patterns evolve. The economic burden placed on the poultry industry because of these diseases may be substantial, as the consequences of muscle abnormalities such as Wooden Breast, White Striping, and Spaghetti Meat can be an impactful reduction in the aesthetic quality of broiler chicken meat and possibly live performance through impact on increased mortality.

Severe cases of these diseases, for example Wooden Breast syndrome, can be discovered through manual palpation of the breast muscle post mortem. However, since the underlying etiology and causative mechanisms have been unknown, and further since physical manipulation and/or sacrifice of the animal may be required to make determinations of disease status possible, there is a need for viable alternative systems, methods, and compositions to determine the presence of one or more diseases, one or more characteristics, one or more compounds, or combination thereof in poultry while the poultry item is alive.

From a biological standpoint, WBS is characterized by muscle degeneration. The normal muscle proteins-sarcoplasmic and myofibrillar—are diminished and replaced by connective tissue, leading to fibrosis and protein denaturation. This not only affects the texture but also alters the nutritional profile of the meat, often reducing its protein content and increasing fat levels.

The development of WBS is closely linked to the rapid growth rates and high breast muscle yields that have been genetically selected for in modern broiler lines. These production traits, while economically beneficial in terms of growth efficiency, appear to predispose birds to muscle disorders like WBS. Additionally, oxidative stress and localized hypoxia (low oxygen levels) in the muscle tissue are believed to contribute to the onset of the condition.

Recent research from 2024 has provided new insights into the causes and mechanisms behind wooden breast syndrome (WBS) in broiler chickens. A team at the University of Delaware used a cutting-edge technique called spatial transcriptomics to study the gene activity in different cell types within chicken breast muscle tissue. This method allowed them to map which genes are active in specific cells and how these contribute to the development of WBS. One of the most significant findings was the identification of lipid-laden macrophages, also known as foam cells, as key contributors to the disease. These white blood cells, found surrounding veins in the muscle tissue, were shown to disrupt fat metabolism and storage. Their presence and activity appear to trigger the muscle degeneration that characterizes WBS, leading to the firm, yellowish, and rubbery texture of the affected meat.

This discovery not only advances the understanding of WBS but also opens new avenues for potential interventions. By targeting these foam cells or the pathways they influence, researchers hope to develop strategies to reduce the incidence of the disease. Interestingly, the findings may also have implications beyond poultry, potentially informing research into human conditions like obesity, where similar cellular mechanisms are involved.

The economic impact remains substantial, with estimates suggesting that WBS affects up to 5% of broiler chickens in some flocks and costs U.S. poultry producers at least $200 million annually. In addition to financial losses, the condition raises animal welfare concerns, as affected birds often struggle with mobility and are more prone to other health issues. The condition also disrupts global supply chains. Meat processors must invest in detection technologies and quality control measures to prevent affected products from reaching consumers. For example, the U.S. has introduced a non-invasive device called the Certified Quality Reader (CQR), which uses electrical impedance to detect WBS in fillets before they are packaged.

In summary, wooden breast syndrome is a global issue that affects meat quality, animal welfare, and the economic sustainability of poultry production. It underscores the need for balanced breeding strategies, improved management practices, and continued research into the biological mechanisms behind the disorder.

As mentioned, traditionally, WBS is identified by palpating and visually examining the breast muscle of a carcass. Affected fillets feel unusually hard, rubbery, and may appear pale or bulging. White striping-white lines running parallel to muscle fibers—is also a common visual indicator. This method is subjective and labor-intensive but still widely used.

In addition, researchers have developed NIR spectroscopy methods for rapid, non-destructive, and large-scale detection of WBS. Industrial NIR scanners can be integrated into processing lines to detect and grade the severity of the condition based on the light absorption characteristics of the muscle tissue, however this process required that the individuals are slaughtered and done directly on the breast muscle without the skin.

There are no viable alternative systems, methodologies, processes, and/or compositions for determination of muscle abnormalities such as Wooden Breast, White Striping, and Spaghetti Meat in live commercial broiler chickens. Thus, there is a need for new systems, methods, and compositions which determine such abnormalities: (a) more efficiently and objectively, (b) in a higher throughput manner, (c) without destruction of the subject, (d) while allowing for elimination of the subject from one or more genetic breeding programs, and/or (e) while allowing for alternative processing of the subject. Thus, the systems, methodologies, processes, and compositions provided herein provide such abilities to determine such abnormalities more efficiently, in a higher throughput manner, without destruction of the subject, while allowing for elimination of the subject from one or more genetic breeding programs, and/or while allowing for alternative processing of the subject.

### SUMMARY OF THE INVENTION

The technology as disclosed herein includes systems, methodologies, processes, and compositions for detection of disease in a subject using spectroscopy and includes more effective detection of muscular myopathies in commercially farmed animals using infrared spectroscopy, and includes detection of Wooden Breast, White Striping, and/or Spaghetti Meat in broiler chickens utilizing near infrared spectroscopy, whether in the development of a strain of flocks having a reduced occurrence of WBS or in a production processing environment where animals are extracted from the production process.

Implementations of the systems, methodologies, processes, and compositions provided herein include and/or utilize and/or provide for detection of various myopathies including Wooden Breast, White Striping, and/or Spaghetti Meat in live broiler chickens prior processing utilizing near infrared spectroscopy to predict levels of taurine in the breast muscle, which is objective and less labor intensive and does not require euthanasia of the poultry item.

The process as disclosed and claimed herein includes scanning the top left side of the breast area of a live feathered or un-feathered poultry item one or more times with a portable handheld micro-Near-Infrared (Micro-NIR) scanner of a NIR spectrometer using light in the near-infrared spectrum to analyze composition of the breast muscle, where the NIR scanner emits light in Near-Infrared range (approximately 700 to 2500 nanometers). Scanning the area twice was found to be preferred. When the light interacts with the breast, certain wavelengths of infrared light are absorbed and some are reflected based on the composition and properties of the breast muscle. The reflected wavelengths of light is detected by a light sensor of the scanner device, which can be diode based light sensor array or other light sensor technology detector. A computing device or controller device having a processor integral with the scanner device is programmed with software code to capture, generate, and analyze the data representative of the absorbed and reflected wavelengths based on the detected/collected wavelengths by the light sensor to determine presence and concentration of various properties within the scanned breast muscle.

The data representative of the determined presence and concentration of properties is output from the NIR scanner device for absorption wavelengths between approximately 908 nm and 1676 nm range, and is input into a Taurine scoring model for determining Taurine levels of the breast tissue of the live bird, where the model outputs data representative of the Taurine levels in a breast tissue of a live bird to a prediction model that predicts whether Woody Breast is present for the breast tissue based on the Taurine level, where higher levels of Taurine are associated with more severe cases of Woody Breast as discovered during the research process as evidenced by the data presented herein.

Selectively tracking a scanned bird for later genetic/genomic evaluation and adding the data representative of the corresponding scanned Taurine Levels and data representative of the genetic/genomic evaluation to the genetic/genomic evaluation models, which determines a breeding value and other performance/production traits that are utilized in an economic profit index is utilized to thereby select individual poultry animals with a lower predisposition to Woody Breast (or lower Taurine levels based on the NIR prediction model), while maintaining or improving other economically relevant traits.

Genetic evaluation over a population has confirmed that there is an underlying genetic basis for the meat quality defects, which is indicated by and correlated to the Taurine level. The heritability based on predicted Taurine level ranges from .32 to .52 depending on the pure line measured and indicates that genetic selection utilizing this process is effective for lowering the propensity of Woody Breast through selective breeding. The heritability predictor has also been correlated with palatability testing. With the process and system as disclosed and claimed herein it is possible to utilize wavelength absorption data through spectroscopy from a Micro-NIR device when used to scan the top side of the breast of a live bird to predict levels of taurine in the breast muscle which has been directly correlated and linked to meat quality characteristics causing Woody Breast and White Stripes in poultry, which is used for breeding program selection and selection during production processing.

While the genetic background of Woody Breast and other meat defects are not completely understood, the current attribute relating to Taurine levels and the process for predicting the presence of Woody Breast based on Taurine levels is an approach that has been validated through finding in gene discovery that highlights pathways/variants/proteins that are known in chicken and other species to modulate myopathies. The findings of the systems, methodologies, processes, and compositions as disclosed and claimed herein, has been validated and confirm that the findings on the association between Taurine predicted levels using NIR wavelengths and observed “Woody Breast”. The findings supportive of the systems, methodologies, processes and compositions show that for each unit decrease in Taurine, a 14% decrease in the incidence of severe Woody Breast is observed (adjusted R-squared: .69), which confirms the efficacy and utility of the technology as disclosed and claimed herein.

Strong evidence has been found that it is possible to utilize wavelength absorption data from Micro-NIR device when used on the top side part of the breast of live birds, whether feathered or de-feathered, to predict levels of taurine in the breast muscle, which is described to be directly linked to meat quality characteristics (wooden breast and white stripes) in poultry.

The systems, methodologies, processes, and compositions disclosed herein also provide for ability to select pedigree birds for improved meat quality while having their own information versus the utilization of data from dissected relatives. This provides a higher volume and more objective data to be used for selection decisions and therefore faster genetic progress. Other features and advantages of the invention will be apparent from the following specification taken in conjunction with the following Figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

To understand the present invention, it will now be described by way of example, with reference to the accompanying drawings in which:

FIG. 1A, is an illustration of NIR Spectroscopy Scanning and Selection Process;

FIG. 1B, is a graphical illustration of heritability of Taurine levels;

FIG. 1C, is an illustration of association between Taurine levels and NIR wavelength;

FIG. 1D, is an illustration of levels of Taurine by Woody Breast category;

FIG. 1E, is a correlation matrix for 190 samples for aa profile of breast meat;

FIG. 1F, is an illustration of the micro-NIR scanner with integrated central processor unit and light sensor communicably coupled with a secondary computing system for analysis and selection.

FIG. 2, is a Scree plot of principal components including 19 aa for 190 breast samples;

FIG. 3, is an illustration of the contribution of each amino acid to estimated principal components;

FIG. 4, is a Biplot of variables in respect to principal components with relative importance (cos2);

FIG. 5, is a graph showing wavelengths significant in the regression/classification analysis of different traits associated to meat quality (ridge, Wooden Breast (WB) and Taurine), based on the Support Vector Machine algorithm;

FIG. 6, is an illustration of the levels of Taurine by Woody Breast Score (WB_D);

FIG. 7, is a Spearman Correlation;

FIG. 8, is a Biplot of variable in respect to principal components including amino acids measured on breast tissue and woody breast scores;

FIG. 9, is an illustration of the Correlations;

FIG. 10, is the R Output: Predictive ability based on feature selection type and model (LM: Linear model; PLS: Partial Least Squares; SVM: Support Vector Machine; RF: Random Forest; Enet: Elastic Net);

FIG. 11, is a flow diagram for processing using NIR detection;

FIG. 12, is an example NIR output for 146 chickens; absorption of each NIR wavelength by chicken;

FIG. 13, is documentation for Taurine predicted values for 146 birds as described herein;

FIG. 14, is the validation data 3.14;

FIG. 15, is the validation Data 3.28;

FIG. 16, is the pooled 3.14 and 3.28 data results;

FIG. 17, is the follow on subsequence lab analysis for pooled data;

FIG. 18, is an illustration of relative importance of variables for prediction;

FIG. 19, is the pooled data modeled for rigidity; and

FIG. 20, is the pooled data modeled for pale.

While the technology as disclosed is susceptible to various modifications and alternative forms, specific implementations thereof are shown by way of example in the drawings and will herein be described in detail. It will be understood, however, that the drawings and detailed description presented herein are not intended to limit the disclosure to the particular implementations as disclosed, but on the contrary, the intention is to cover all modifications, equivalents, and alternatives falling within the scope of the present technology as disclosed and as defined by the appended claims.

### DETAILED DESCRIPTION

### Overview

There is a need for new systems, methods, processes, and compositions which can detect muscle disorders in a live subject using spectroscopy, and particularly for more effective detection of muscular myopathies in commercially farmed animals, including detection of Wooden Breast, White Striping, and/or Spaghetti Meat in commercial broiler chickens. Thus, there is a need for new systems, methods, and compositions which determine such abnormalities: (a) more efficiently and objectively, (b) in a higher throughput manner, (c) without destruction of the subject, (d) while allowing for elimination of the subject from one or more genetic breeding programs, and/or (e) while allowing for alternative processing of the subject. Thus, the systems, methods, and compositions provided herein provide such abilities to determine such abnormalities more efficiently, in a higher throughput manner, without destruction of the subject, while allowing for elimination of the subject from one or more genetic breeding programs, and/or while allowing for alternative processing of the subject.

### Definitions

To the extent necessary to provide descriptive support, the subject matter and/or text of the appended claims is incorporated herein by reference in their entirety.

The headings provided herein are solely for ease of reference and are not limitations of the various aspects or aspects of the disclosure, which can be had by reference to the specification as a whole.

It will be understood by all readers of this written description that the exemplary implementations described and claimed herein may be suitably practiced in the absence of any recited feature, element or step that is, or is not, specifically disclosed herein.

It must be noted that as used herein and in the appended claims, the singular forms “a,” “an,” and “the” include the plural reference unless the context clearly dictates otherwise; for example, “an element” is a reference to one or more elements and includes equivalents thereof known to those skilled in the art. Similarly, for another example, a reference to “a step” or “a means” is a reference to one or more steps or means and can include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that can be construed to express approximation should be so understood unless the context clearly dictates otherwise. As such, the terms “a” (or “an”), “one or more,” and “at least one” can be used interchangeably herein. Singular words should be read as plural and vice versa and masculine as feminine and vice versa, where appropriate, and alternative implementations do not necessarily imply that the two are mutually exclusive.

Furthermore, “and/or” where used herein is to be taken as specific disclosure of each of the specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

It will be further understood that use of the word “can” and/or “may” will be understood to refer to the active, and enabling, dictionary meanings of “is able,” “be able”, “to know,” “be able to through acquired knowledge or skill,” “to know how to do something,” and/or “to have the ability to do something”; and not understood to intend a sense of “maybe” or permissiveness.

Reference in the specification to “one embodiment” or “an embodiment”; “one implementation” or “an implementation” means that a particular feature, structure, or characteristic described in connection with the embodiment or implementation is included in at least one embodiment or implementation of the present invention. The appearances of the phrase “in one embodiment,” or “in an embodiment,” or “in one implementation,” or “in an implementation” in various places in the specification are not necessarily all referring to the same embodiment or the same implementation, nor are separate or alternative embodiments or implementations mutually exclusive of other embodiments or implementations. The words “embodiment(s)” and “implementation(s)” can be used interchangeably based on context.

It is understood that wherever aspects are described herein with the language “comprising,” otherwise analogous aspects described in terms of “consisting of” and/or “consisting essentially of” are also provided.

Unless defined otherwise, technical and scientific terms used herein have the same meaning as commonly understood by one of ordinary skill in the art to which this disclosure is related. Although any methods, materials, features and/or components, similar or equivalent to those described herein can also be used in the practice or testing of various implementations of the present disclosure, exemplary methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present disclosure is not entitled to antedate such publication by virtue of prior invention. Further, the dates of publication provided may be different from the actual publication dates, which may need to be independently confirmed.

Numeric ranges are inclusive of the numbers defining the range. Even when not explicitly identified by “and any range in between,” or the like, where a list of values is recited, e.g., 1, 2, 3, or 4, the disclosure specifically includes any range in between the values, e.g., 1 to 3, 1 to 4, 2 to 4, etc. Where a range of values is provided, it is understood that each intervening value, to the tenth of the unit of the lower limit unless the context clearly dictates otherwise, between the upper and lower limit of that range and any other stated or intervening value in that stated range, is encompassed within the present disclosure. The upper and lower limits of these smaller ranges that may independently be included in the smaller ranges is also encompassed within the present disclosure; subject to any specifically excluded limit in the stated range. Where the stated range includes one or both of the limits, ranges excluding either or both of those included limits are also included in the present disclosure.

As will be apparent to those of skill in the art upon reading this disclosure, each of the individual implementations described and illustrated herein has discrete components and features which may be readily separated from or combined with the features of any of the other implementations without departing from the scope or spirit of the present disclosure.

It is also to be understood that the terminology used herein is for the purpose of describing particular implementations only and is not intended to be limiting of the scope of the present disclosure.

The terms defined immediately below are more fully defined by reference to the specification and drawing in its entirety.

“IR” refers to infrared wavelengths of the electromagnetic spectrum.

“NIR” and “Near IR” refer to near infrared wavelengths of the electromagnetic spectrum, and may be used interchangeably herein.

“Woody Breast” and “Wooden Breast” refer to breast muscle myopathies of chickens (or other poultry animal) and may be used interchangeably herein.

“Selection” shall be taken to mean a selection controlled including systems, processes, steps or combinations of steps of a breeding program for producing genetic gain, including the collective design and/or implementation of the breeding program and any intermediate steps. It is understood that selection requires a determination based on a defined selection criteria, of one or more individuals in a population that are to be parents and ultimately ancestors, thereby producing a desired genetic gain.

“Ancestor” means an individual having a genetic contribution to a current population. “Ancestor” is a function of pedigree, the determination of which does not require prior knowledge of a particular trait or combination of traits present in the current population and its current progenitors.

“Breeding objective” refers to a goal of a selection program. “Breeding objective” may be determined by weighted combination of traits defining and aggregate breeding value of an animal.

“Breeding value” means the genetic value of an individual as a parent in a breeding program and, more particularly, the effect of an individual's genes or genetic markers when considered in isolation or in combination (aggregate breeding value) on performance against a selection criterion or selection criteria.

Implementation(s) of the present invention are discussed below with reference to the Figures. However, those skilled in the art will readily appreciate that the detailed description given herein with respect to these Figures is for explanatory purposes as the present invention extends beyond these limited implementations. For example, it will be appreciated that those skilled in the art will, in light of the teachings of the present invention, recognize a multiplicity of alternate and suitable approaches, depending upon the needs of the particular application, to implement the functionality of any given detail described herein, beyond the particular implementation choices described and shown. That is, there are modifications and variations of the present invention that are too numerous to be listed but that all fit within the scope of the present invention.

According to the implementation(s) of the present technology as disclosed, various views are illustrated in Figures throughout, and with like reference numerals being used consistently throughout to refer to like and corresponding parts of the technology for all of the various views and Figures of the drawing. Also, please note that the first digit(s) of the reference number for a given item or part of the technology should correspond to the Figure (FIG.) number in which the item or part is first identified.

As is evident from the foregoing and following description, certain aspects of the present implementation(s) are not limited by the particular details of the examples illustrated herein, and it is therefore understood that other modifications and applications, or equivalents thereof, will be apparent to those skilled in the art. It is accordingly intended that the claims shall cover all such modifications and applications that do not depart from the scope of the present implementation(s). Accordingly, the specification and drawings are to be regarded in an illustrative rather than a restrictive sense.

### DESCRIPTION

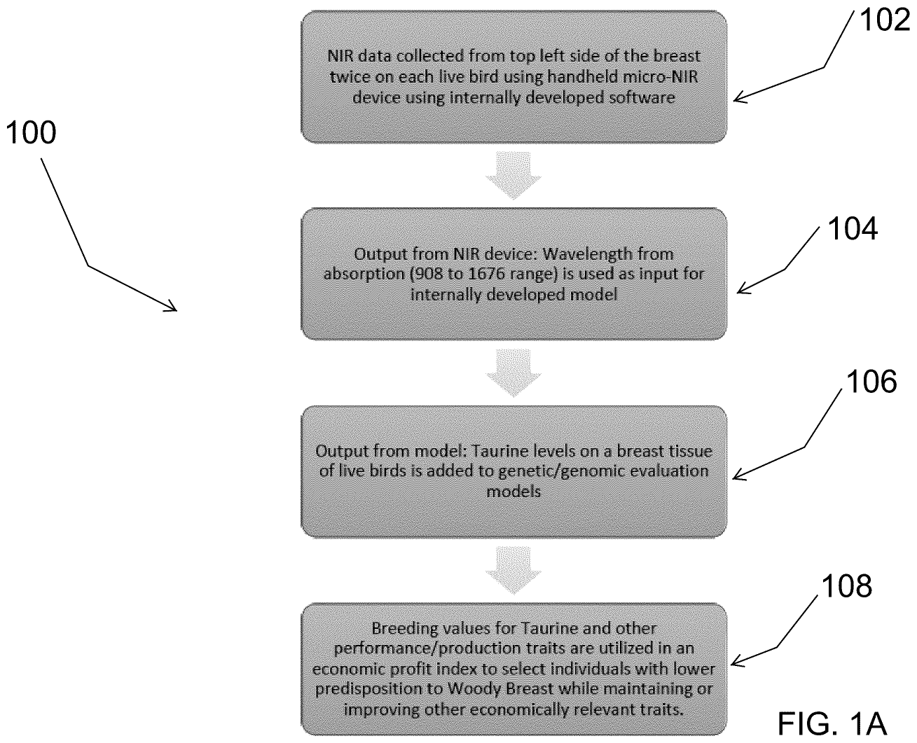

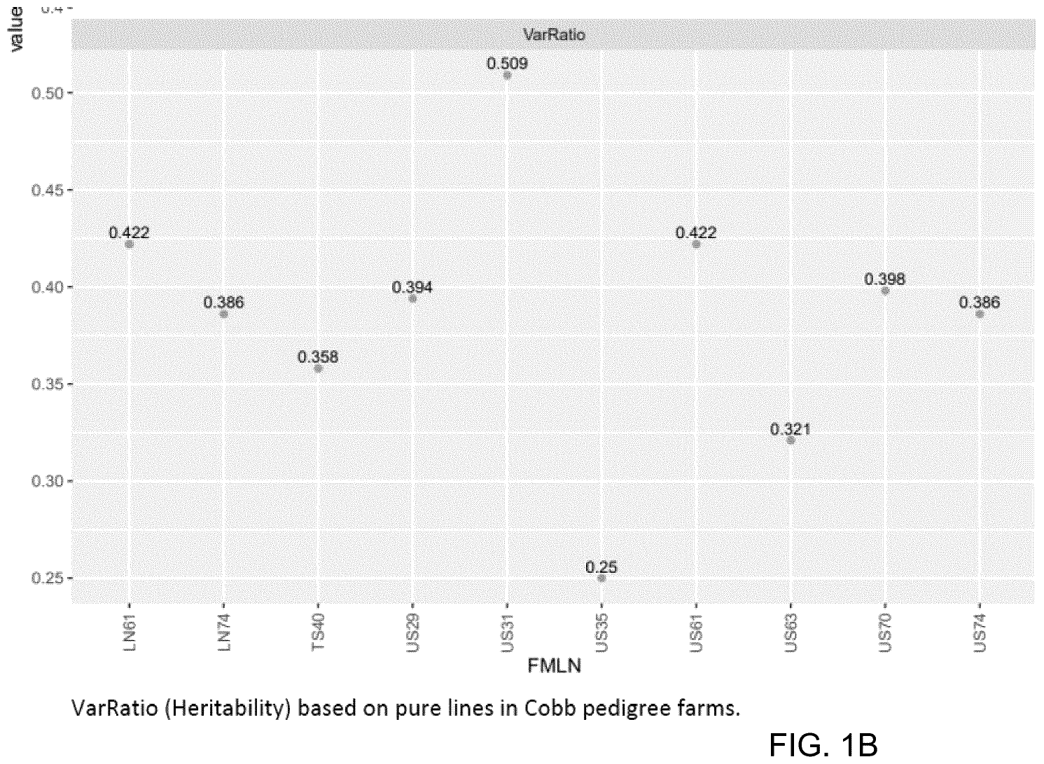

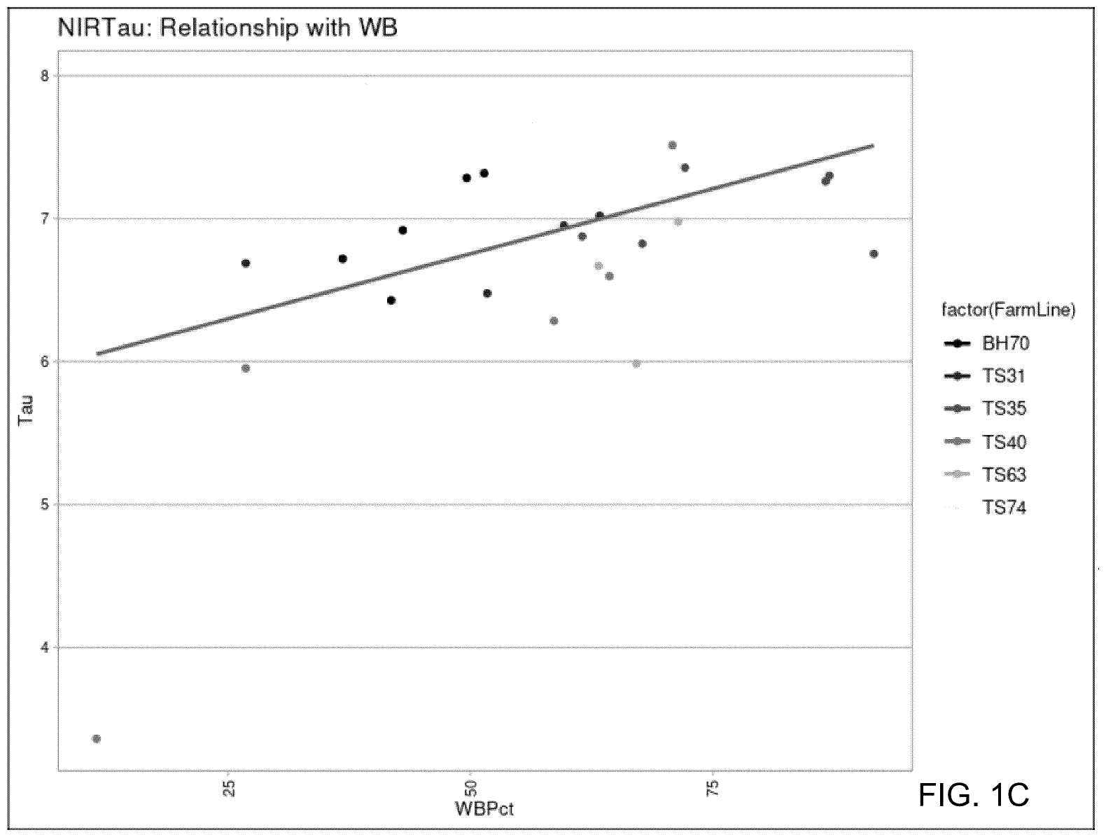

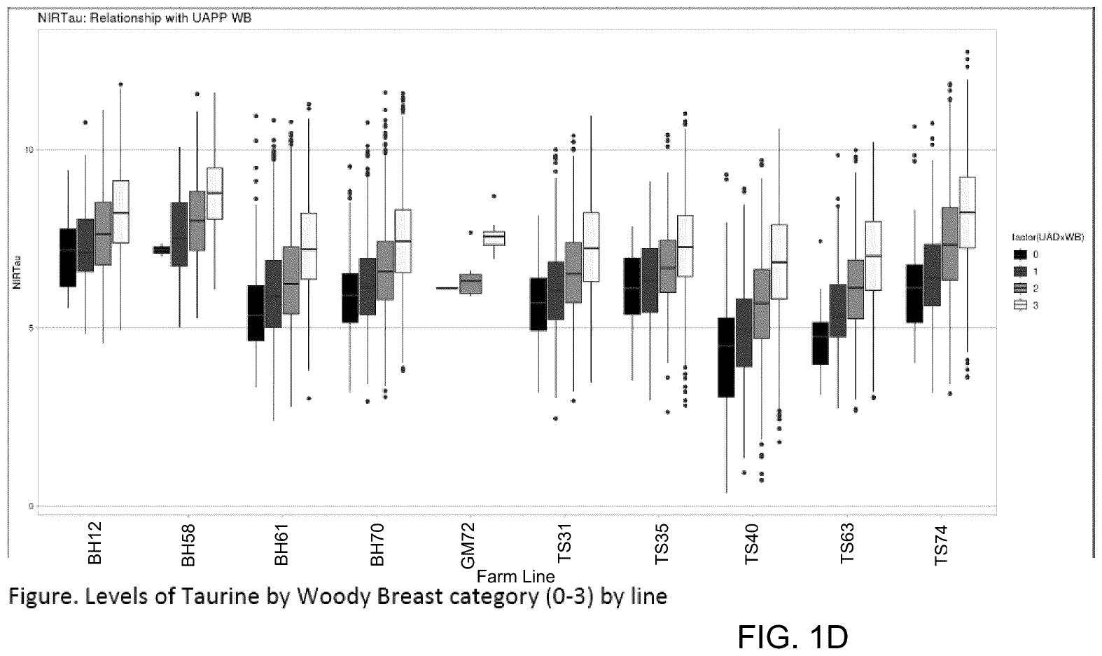

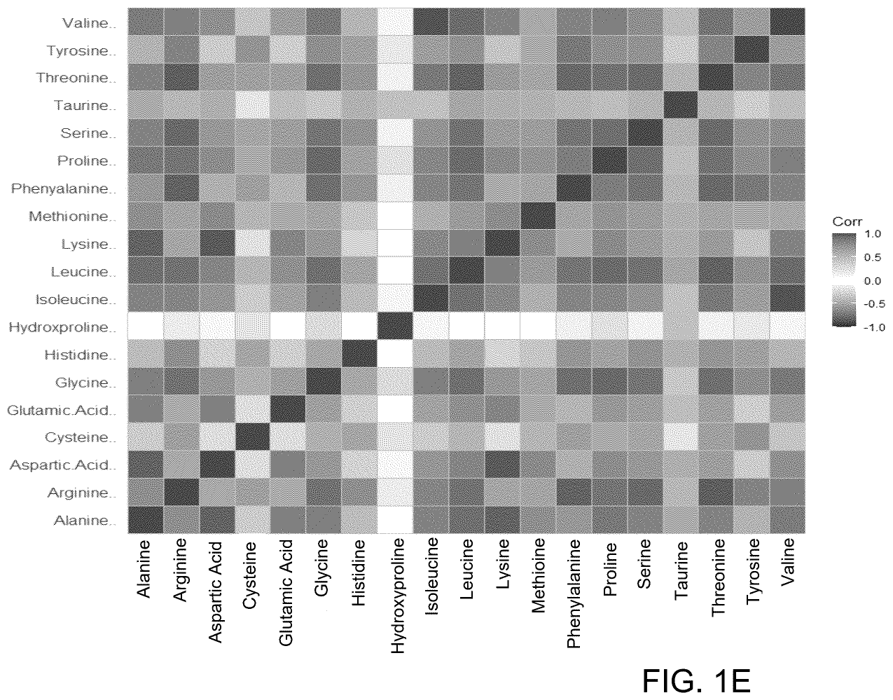

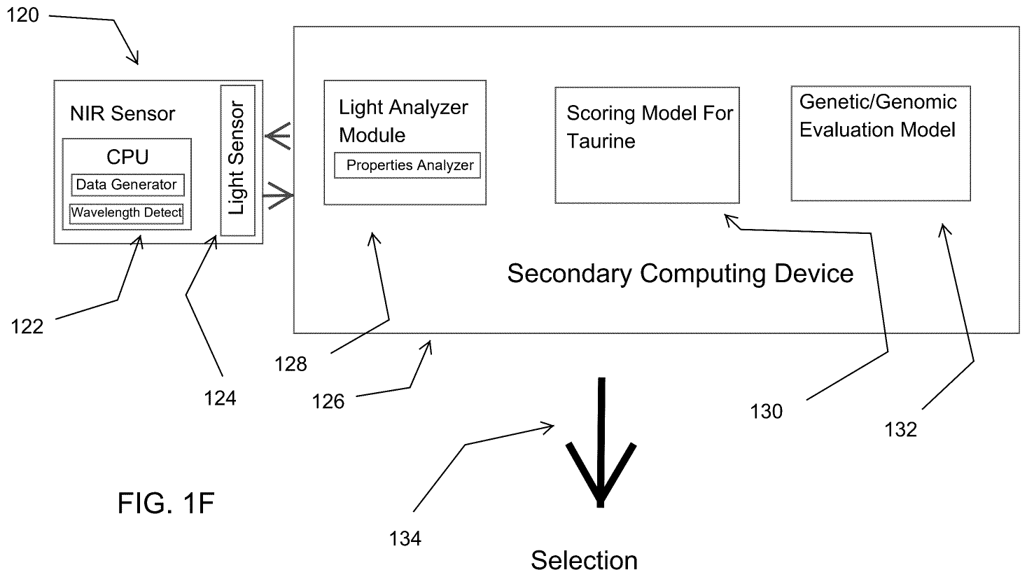

Genetic evaluation over a population has confirmed that there is an underlying genetic basis for the meat quality defects, which is indicated by and correlated to the Taurine level. The heritability predictor based on Taurine level ranges from .32 to .52 depending on the pure line measured and indicates that genetic selection utilizing this process is effective for lowering the propensity of Woody Breast through selective breeding, refer to FIG. 1B, which is an graphical illustration of heritability. The heritability predictor has also been correlated with Woody breast scores of slaughtered carcasses using palatability, visual and other testing. With the process and system as disclosed and claimed herein it is possible to utilize wavelength absorption data through spectroscopy from a Micro-NIR device when used to scan the top side of the breast of a live bird to predict levels of taurine in the breast muscle which has been directly correlated and linked to meat quality characteristics causing Woody Breast and White Stripes in poultry, which is used for breeding program selection and removal of individuals with lower genetic merit for meat quality (or higher breeding values for Taurine). The process of scanning as disclosed and claimed herein for selection and removal of a poultry subject from production processing.

While the genetic background of Woody Breast and other meat defects are not completely understood, the current attribute relating to Taurine levels and the process for predicting the presence of Woody Breast based on Taurine levels is an approach that has been validated through finding in gene discovery that highlights pathways/variants/proteins that are known in chicken and other species to modulate myopathies. The findings of the systems, methodologies, processes, and compositions as disclosed and claimed herein, has been validated and confirm that the findings on the association between Taurine predicted levels using NIR wavelengths and observed “Woody Breast”. The findings supportive of the systems, methodologies, processes and compositions show that for each unit decrease in Taurine, a 14% decrease in the incidence of severe Woody Breast is observed (adjusted R-squared: .69), which confirms the efficacy and utility of the technology as disclosed and claimed herein, please refer to FIGS. 1C and 1D. Therefore, it has been validated that wavelength absorption data from a Micro-NIR device taking scans of the top side part of the breast of a live bird has sufficient efficacy to predict levels of Taurine and hence predict the likelihood of Woody Breast.

As part of the validation trials, data from NIR scans was correlated with the presence or absence of Woody Breast through collection of visual and palpation scores, amino acid chemical profiles and FOSS chemical profiles (fat % and collagen %) for 190 birds. The birds measured belonged to three different pure lines from pedigree populations.

Strong evidence has been found that it is possible to utilize wavelength absorption data from Micro-NIR device when used on the top side part of the breast of live birds to predict levels of taurine in the breast muscle, which is described to be directly linked to meat quality characteristics (wooden breast and white stripes) in poultry. The systems, methodologies, processes, and compositions disclosed herein also provide for ability to select pedigree birds for improved meat quality while having their own information versus the utilization of data from dissected relatives. This provides a higher volume and more objective data to be used for selection decisions and therefore faster genetic progress. Other features and advantages of the invention will be apparent from the following specification taken in conjunction with the following detailed description of the Figures.

Referring to FIG. 1A, the process 100 as disclosed and claimed herein is illustrated, which includes scanning twice 102 the left side of a live feathered or un-feathered poultry item with a portable handheld micro-Near-Infrared (Micro-NIR) scanner 120 of a NIR spectrometer using light in the near-infrared spectrum to analyze composition of the breast muscle, where the NIR scanner emits light in Near-Infrared range (approximately 700 to 2500 nanometers). When the light interacts with the breast, certain wavelengths are absorbed, and some are reflected based on the composition and properties of the breast muscle. The reflected wavelength of light is detected by a light sensor 124 of the scanner device, which can be diode based light sensor array or other light sensor technology detector. A computing device or controller device having a processor 122 integral with the scanner device is programmed with software code to capture and analyze the data 122 representative of the absorbed and reflected wavelengths based on the collected wavelengths by the light sensor to determine presence and concentration of various properties within the scanned breast muscle.

The data representative of the determined presence and concentration of properties 128 is output from the NIR scanner device for absorption wavelengths between approximately 908 nm and 1676 nm range, See 104, and is input into a Taurine scoring model 104 for determining Taurine levels of the breast tissue of the live bird, where the model 130 is programmed in software and executing on a secondary computing device 130 and outputs data representative of the Taurine levels 106 on a breast tissue of a live bird to a prediction model that predicts whether Woody Breast is present for the breast tissue based on the Taurine level. Selectively tracking a scanned bird for later genetic/genomic evaluation and adding the data representative of the corresponding scanned Taurine Levels and data representative of the genetic/genomic evaluation to the genetic/genomic evaluation models, 132 which determines a breeding value and other performance/production traits 108 that are utilized in an economic profit index to thereby select 134 individual poultry animals with a lower predisposition to Woody Breast, while maintaining or improving other economically relevant traits. The systems, methods, processes and compositions disclosed and claimed herein provide for the ability to select pedigree birds for improved meat quality while having their own information versus the utilization of data from dissected carcasses of relatives. This provides a high volume and for more objective data to be used for the selection decisions and therefore more efficient genetic progress.

Genetic evaluation over a population has confirmed that there is an underlying genetic basis for the meat quality defects, which is indicated by and correlated to the Taurine level. The heritability predictor based on Taurine level ranges from .32 to .52 depending on the pure line measured and indicates that genetic selection utilizing this process is effective for lowering the propensity of Woody Breast through selective breeding. With the process and system as disclosed and claimed herein it is possible to utilize wavelength absorption data through spectroscopy from a Micro-NIR device when used to scan the top side of the breast of a live bird to predict levels of taurine in the breast muscle which has been directly correlated and linked to meat quality characteristics causing Woody Breast and White Stripes in poultry, which is used for breeding program selection and selection during production processing.

While the genetic background of Woody Breast and other meat defects are not completely understood, the current attribute relating to Taurine levels and the process for predicting the presence of Woody Breast based on Taurine levels is an approach that has been validated through finding in gene discovery that highlights pathways/variants/proteins that are known in chicken and other species to modulate myopathies. The findings of the systems, methodologies, processes, and compositions as disclosed and claimed herein, has been validated and confirm that the findings on the association between Taurine predicted levels using NIR wavelengths and observed “Woody Breast”. The findings supportive of the systems, methodologies, processes and compositions show that for each unit decrease in Taurine, a 14% decrease in the incidence of severe Woody Breast is observed (adjusted R-squared: .69), which confirms the efficacy and utility of the technology as disclosed and claimed herein.

### Validation of the Process and System was Performed by the Use of Micro-NIR Information on Live Broilers to Predict Level of Amino Acid Levels in Breast Muscle.

Results—NIR information is used to predict taurine levels in the breast tissue of live birds. High taurine levels, as validated, are directly associated with higher scores of woody breast. Data shows that high predicted taurine levels are associated with higher scores for Wooden Breast. Individual bird taurine predictions can be used for family selection towards lower susceptibility to woody breast in a more objective way and not requiring birds to be slaughtered. In addition, birds on the extreme right side of the distribution taurine level can be removed from the pedigree population due to high probability of meat quality issue which also correlates to welfare.

Strong evidence for utilization of wavelength absorption data from Micro-NIR device, used on the top side part of the breast of live birds, to predict levels of Taurine in the breast muscle, and the meat quality characteristics of wooden breast and white stripes in poultry. Such information, in certain implementations, is used to direct genetic improvement for poultry lines.

### Methods

Data on NIR, woody breast (WB) visual/palpation scores, amino acid profile and FOSS (fat % and collagen %) was collected on 190 birds. Birds measured belonged to three different pure lines from pedigree populations.

### Data Gathered

Amino-acids available: Alanine, Arginine, Aspartic acid, Cysteine, Glutamic Acid, Glycine, Histidine, Hydroxyproline, Isoleucine, Leucine, Lysine, Methionine, Phenylalanine, Proline, Serine, Taurine, Threonine, Tyrosine and Valine.

TABLE 1
Micro-NIR: 125 Wavelengths

| | | | | | |
|---|---|---|---|---|---|
| NIR.908 | NIR.914 | NIR.920 | NIR.926 | NIR.932 | NIR.939 |
| NIR.945 | NIR.951 | NIR.957 | NIR.963 | NIR.970 | NIR.976 |
| NIR.982 | NIR.988 | NIR.994 | NIR.1001 | NIR.1007 | NIR.1013 |
| NIR.1019 | NIR.1025 | NIR.1031 | NIR.1038 | NIR.1044 | NIR.1050 |
| NIR.1056 | NIR.1062 | NIR.1069 | NIR.1075 | NIR.1081 | NIR.1087 |
| NIR.1093 | NIR.1100 | NIR.1106 | NIR.1112 | NIR.1118 | NIR.1124 |
| NIR.1131 | NIR.1137 | NIR.1143 | NIR.1149 | NIR.1155 | NIR.1162 |
| NIR.1168 | NIR.1174 | NIR.1180 | NIR.1186 | NIR.1193 | NIR.1199 |
| NIR.1205 | NIR.1211 | NIR.1217 | NIR.1224 | NIR.1230 | NIR.1236 |
| NIR.1242 | NIR.1248 | NIR.1254 | NIR.1261 | NIR.1267 | NIR.1273 |
| NIR.1279 | NIR.1285 | NIR.1292 | NIR.1298 | NIR.1304 | NIR.1310 |
| NIR.1316 | NIR.1323 | NIR.1329 | NIR.1335 | NIR.1341 | NIR.1347 |
| NIR.1354 | NIR.1360 | NIR.1366 | NIR.1372 | NIR.1378 | NIR.1385 |
| NIR.1391 | NIR.1397 | NIR.1403 | NIR.1409 | NIR.1416 | NIR.1422 |
| NIR.1428 | NIR.1434 | NIR.1440 | NIR.1447 | NIR.1453 | NIR.1459 |
| NIR.1465 | NIR.1471 | NIR.1477 | NIR.1484 | NIR.1490 | NIR.1496 |
| NIR.1502 | NIR.1508 | NIR.1515 | NIR.1521 | NIR.1527 | NIR.1533 |
| NIR.1539 | NIR.1546 | NIR.1552 | NIR.1558 | NIR.1564 | NIR.1570 |
| NIR.1577 | NIR.1583 | NIR.1589 | NIR.1595 | NIR.1601 | NIR.1608 |
| NIR.1614 | NIR.1620 | NIR.1626 | NIR.1632 | NIR.1639 | NIR.1645 |
| NIR.1651 | NIR.1657 | NIR.1663 | NIR.1670 | NIR.1676 | |

Woody-Breast scores: (0 (absence), 1, 2 and 3 (severe WB)) for Live (L) and processed bird (D)

### Results

Amino acids-Hydroxyproline, Taurine and Cysteine show the lowest standard deviations (0.01, 0.02 and 0.03, respectively). In addition, Hydroxyproline is not associated with others amino acid contents, except for Taurine (See FIG. 1E). Taurine is a non-proteogenic aa reported to accumulate during the start process of breast meat oxidative stress, specifically associated with white stripes. Given the higher Variation, Taurine was determined to be a novel target for a meat quality predictive model. FIG. 1E illustrates a correlation matrix for 190 samples for aa profile of breast meat.

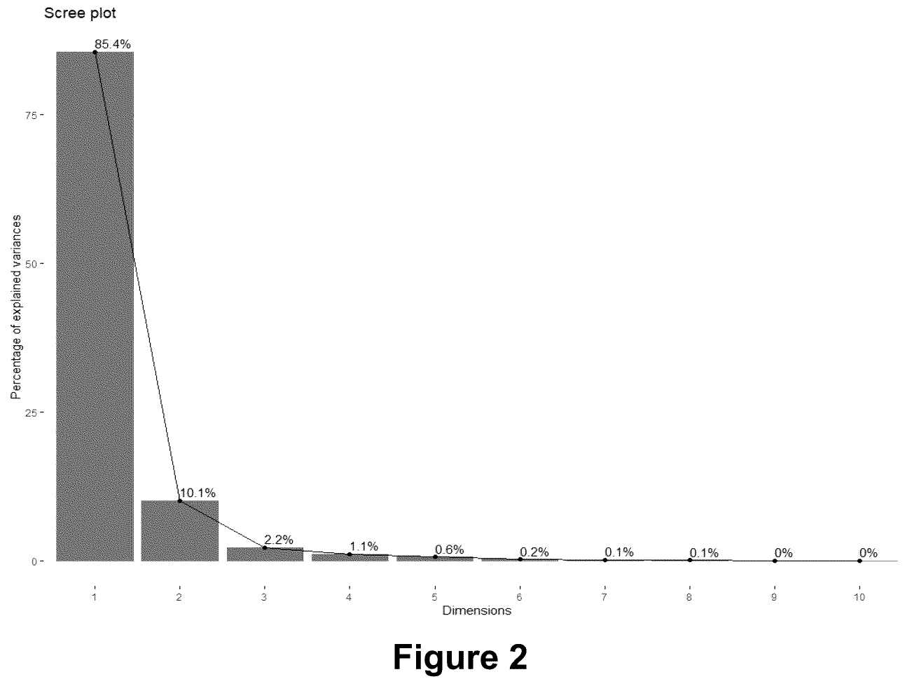

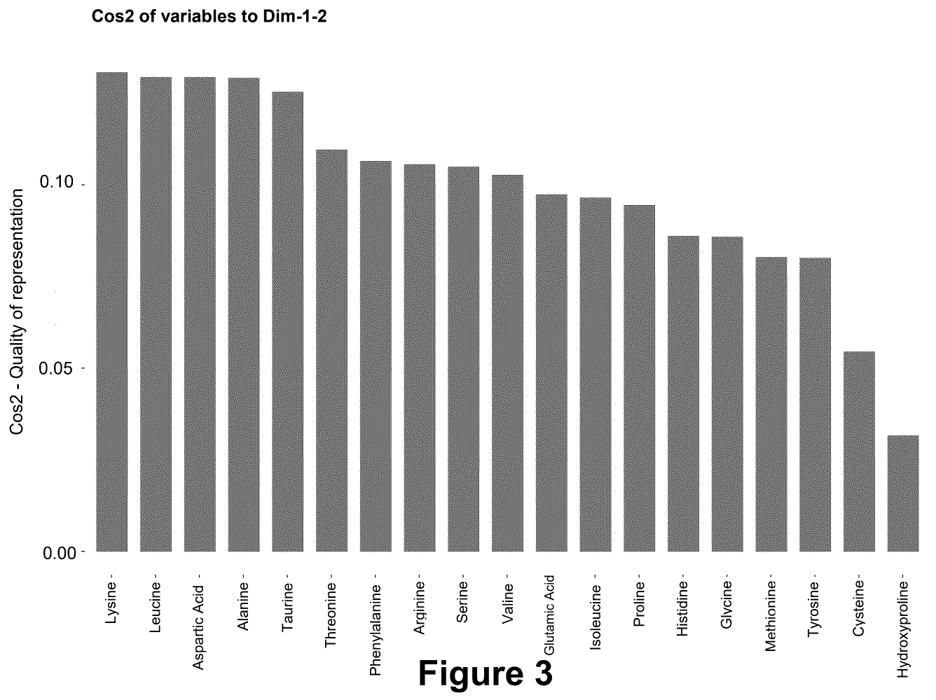

In Principle Component Analysis (PCA), the first 2 dimensions captured 95.5% of the total variance in the dataset. Based on the square cosine, Hydroxyproline and Taurine are the least represented by the first two principal components. Referring to FIG. 2, provided is an illustration of a Scree plot (a graph utilized in principle component analysis and factor analysis to visually determine the optimal number of components or factors to retain) of principal components including 19 aa for 190 breast samples. Referring to FIG. 3, provide is a graphical illustration of the contribution of each amino acid to estimated principal components.

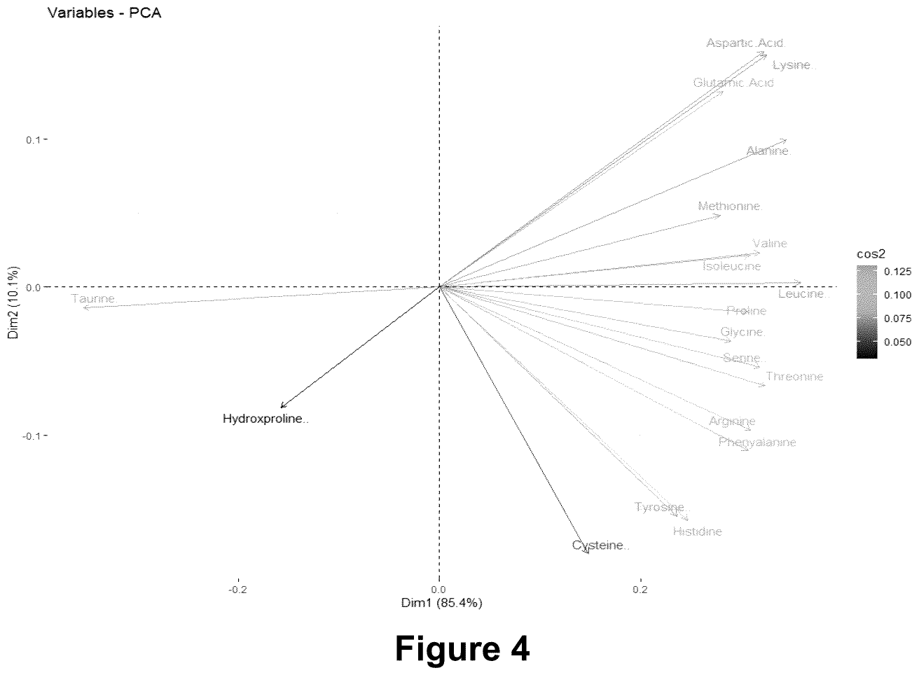

Referring to FIG. 4, provided is a graphical illustration of a biplot of variables in respect to principal components with relative importance (cos2). The biplot illustrates a graphical representation for PCA to illustrate the relationship between observations and variables within a dataset. This graphical representation combines two types of information, that is, scores (representing observations) and loadings (representing variables) on a single plot. This graphical representation provides for a comprehensive view of how variables contribute to the principal components and how observations relate to each other and the variables.

Further evaluations focusing on Taurine, showed a strong association between Taurine levels and woody breast severity (r=.74); even considering the subjective nature of the calls for WB, the highest taurine levels were only encountered for the most severely affected breast muscles. Although not with as strong association with Woody Breast, other amino acids showed negative correlation with Taurine levels, and in certain implementations are further used in a predictive model.

### Near Infrared Detection Methods

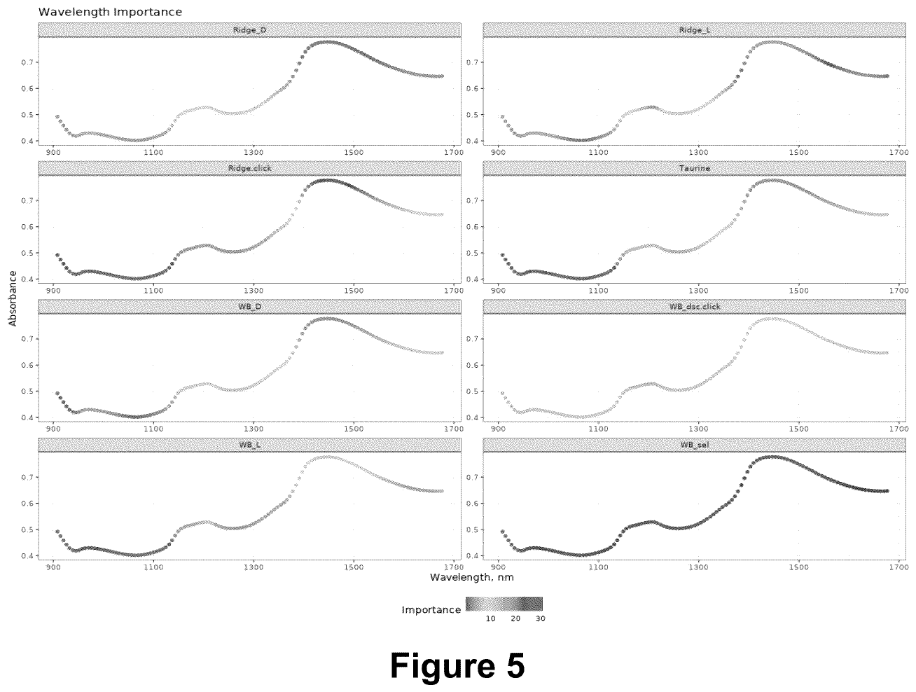

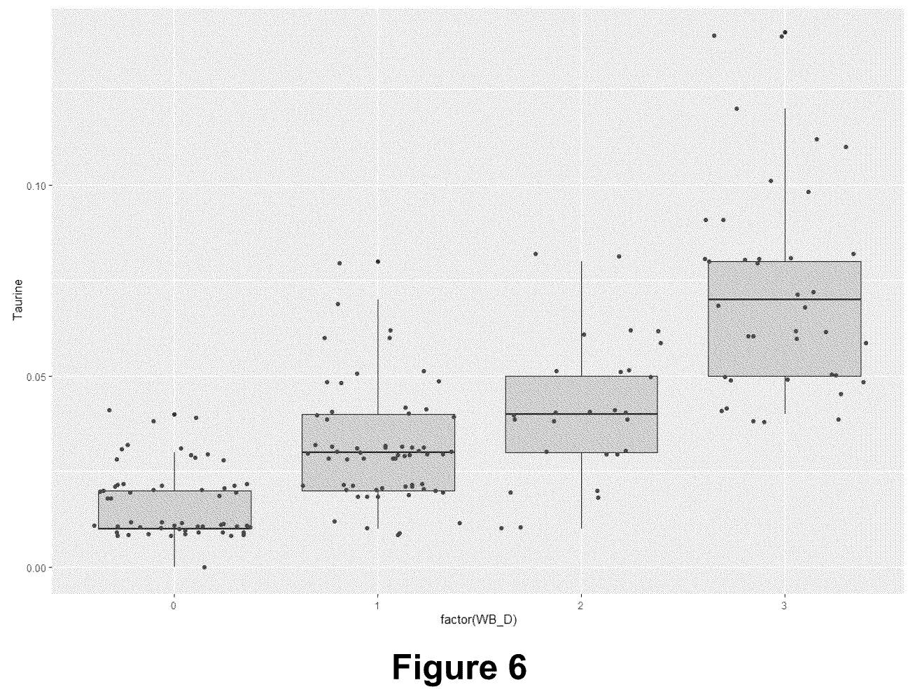

Referring to FIG. 5, provided is a graphical illustration showing wavelengths significant in the regression/classification analysis, based on the Support Vector Machine algorithm. The results indicate certain wavelengths are more important than others to predict levels of Taurine. Support Vector Machine (SVM), which is a supervised machine learning algorithm used for classification and regression tasks. SVMs work by finding the optimal hyperplane that separates data points into different classes or predicts a continuous outcome. The input data is preprocessed and potentially transformed using kernel functions to handle complex relationships. SVM algorithms search for the hyperplane that maximizes the margin between classes, ensuring a robust separation of data points. Once the hyperplane is found, it's used to classify new, unseen data points or predict continuous values based on their position relative to the hyperplane. This regression/classification process allows for the identification of certain wavelengths that are more important than others for the prediction of levels of Taurine and hence a prediction for likelihood of Woody Breast. FIG. 6, illustrates graphically how Levels of Taurine are grouped by Woody Breast Scores, where higher Woody Breast scores have higher levels of Taurine.

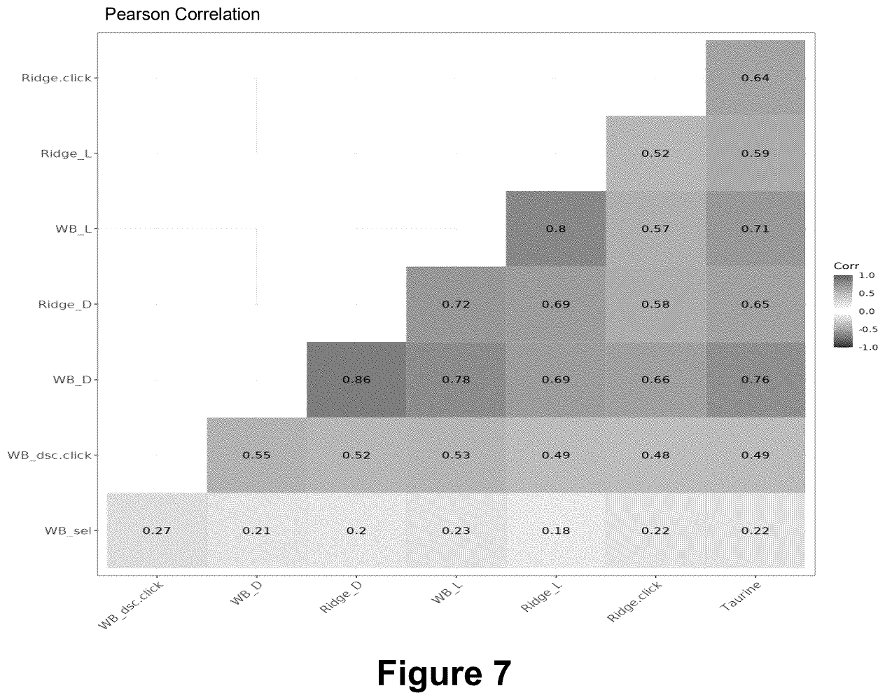

Referring to FIG. 7, provided is a graphical illustration of Pearson Correlation (also noted as r), which is a statistical measure that quantifies the strength and direction of the linear relationship between two variables. It indicates the strength and direction of association, with values ranging from −1 to +1. A value of +1 indicates a perfect positive monotonic relationship, −1 indicates a perfect negative monotonic relationship, and 0 indicates no monotonic relationship. Pearson correlation, also known as Pearson's r, is a statistical measure that quantifies the linear relationship between two continuous variables. It assesses both the strength and direction of the relationship, ranging from −1 to +1. A value of +1 indicates a perfect positive linear relationship, −1 indicates a perfect negative linear relationship, and 0 indicates no linear relationship. Pearson correlation specifically looks for a straight-line relationship between the variables. The magnitude of the correlation coefficient (absolute value) indicates the strength of the relationship. A value closer to 1 (or −1) suggests a strong relationship, while a value closer to 0 suggests a weak relationship. The sign of the correlation coefficient (+or −) indicates the direction of the relationship. A positive sign means that as one variable increases, the other also tends to increase. A negative sign means that as one variable increases, the other tends to decrease.

Intuitively, the Pearson correlation between two variables will be high when observations have a similar (or identical for a correlation of 1) rank (i.e. relative position label of the observations within the variable: 1st, 2nd, 3rd, etc.) between the two variables, and low when observations have a dissimilar (or fully opposed for a correlation of −1) rank between the two variables. As can be seen, there is a strong correlation between Woody Breast and Taurine as detected by the NIR device.

TABLE 2
Level of Taurine by group lsmeans and tukey test significance.

| Trait | Group | LSMEAN | SE | TEST | Comment |
|---|---|---|---|---|---|
| Line | 63 | 0.0412821 | 0.0041846 | a | No line effect |
| Line | 31 | 0.0359302 | 0.002818 | a | |
| Line | 74 | 0.0348387 | 0.0033189 | a | |
| Ridge_D | 1 | 0.0589552 | 0.0024592 | a | Higher Taurine |
| | | | | | on Ridge = 1 |
| Ridge_D | 0 | 0.02425 | 0.0018375 | b | |
| Ridge_L | 1 | 0.0548052 | 0.0024249 | a | Higher Taurine |
| | | | | | on Ridge = 1 |
| Ridge_L | 0 | 0.024 | 0.0020288 | b | |
| Ridge.click | 3 | 0.0612195 | 0.0032336 | a | Higher Taurine |
| | | | | | level on Ridge = |
| | | | | | 3 |
| Ridge.click | 2 | 0.0419048 | 0.0026086 | b | |
| Ridge.click | 1 | 0.0206024 | 0.0022727 | c | |
| WB_D | 3 | 0.0715385 | 0.0027597 | a | Higher taurine |
| WB_D | 2 | 0.0414815 | 0.0033168 | b | levels on higher |
| WB_D | 1 | 0.0312903 | 0.0021888 | b | woody scores |
| WB_D | 0 | 0.0171186 | 0.0022437 | c | |
| WB_L | 3 | 0.0775 | 0.0036152 | a | |
| WB_L | 2 | 0.0493333 | 0.0026402 | b | |
| WB_L | 1 | 0.0279245 | 0.0024328 | c | |
| WB_L | 0 | 0.02 | 0.0021967 | c | |
| WB_sel | 2 | 0.0866667 | 0.0145693 | a | |
| WB_sel | 1 | 0.08 | 0.0252347 | ab | |
| WB_sel | 0 | 0.0356284 | 0.0018654 | b | |

There is a clear significance of Taurine in association with Woody Breast across the groups based on at least square means analysis and Tukey average test comparison.

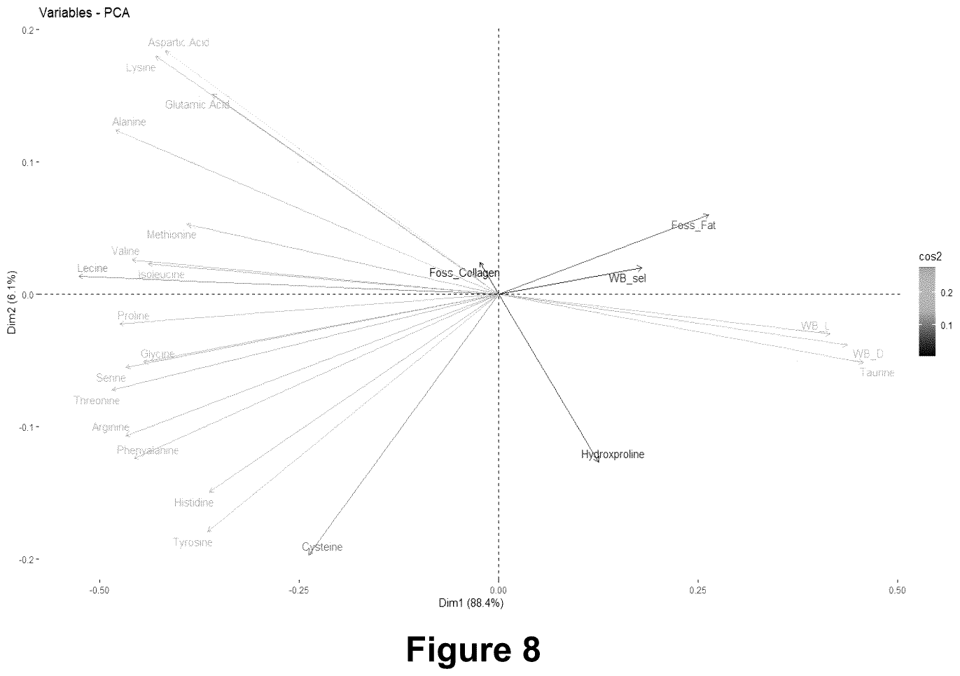

Referring to FIG. 8, provided is a graphical illustration of a biplot of variables in respect to principal components with relative importance (cos2). The biplot illustrates a graphical representation for Variables-PCA to illustrate the relationship between observations and variables within a dataset. This graphical representation combines two types of information, that is, scores (representing observations) and loadings (representing variables) on a single plot. This graphical representation provides for a comprehensive view of how variables contribute to the principal components and how observations relate to each other and the variables.

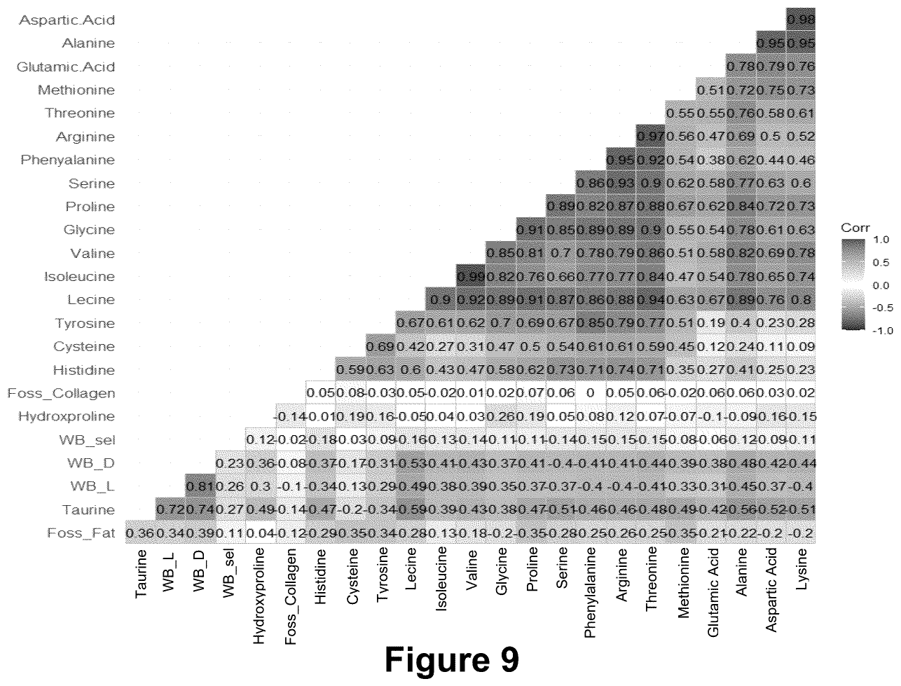

Referring to FIG. 9, provided is a graphical illustration of Pearson Correlation, which is a statistical measure that assesses the monotonic relationship between two variables. It indicates the strength and direction of association, with values ranging from −1 to +1. A value of +1 indicates a perfect positive monotonic relationship, −1 indicates a perfect negative monotonic relationship, and 0 indicates no monotonic relationship. The correlation in this instance is amongst the various amino acids. As part of the validation trials, data from NIR scans was correlated with the presence or absence of Woody Breast through collection of visual and palpation scores, amino acid chemical profiles and FOSS chemical profiles (fat % and collagen %) for 190 birds. The birds measured belonged to three different pure lines from pedigree populations. Amino acids-Hydroxproline, Taurine and Cysteine show the lowest standard deviations (0.01, 0.02 and 0.03, respectively). In addition, Hydroxproline is not associated with others amino acid contents, except for Taurine (See FIG. 1E). Taurine is a non-proteogenic aa reported to accumulate during the start process of breast meat oxidative stress, specifically associated with white stripes. Given the higher Variation, Taurine was determined to be a novel target for a meat quality predictive model. FIG. 1E illustrates a correlation matrix for 190 samples for aa profile of breast meat.

In a Principle Component Analysis (PCA), the first 2 dimensions captured 95.5% of the total variance in the dataset. Based on the square cosine, Hydroxyproline and Taurine are the least represented by the first two principal components. Referring to FIG. 2, provided is an illustration of a Scree plot (a graph utilized in principle component analysis and factor analysis to visually determine the optimal number of components or factors to retain) of principal components including 19 aa for 190 breast samples. Referring to FIG. 3, provide is a graphical illustration of the contribution of each amino acid to estimated principal components.

The current attribute relating to Taurine levels and the process for predicting the presence of Woody Breast based on Taurine levels is an approach that has been validated through finding in gene discovery that highlights pathways/variants/proteins that are known in chicken and other species to modulate myopathies. The findings of the systems, methodologies, processes, and compositions as disclosed and claimed herein, has been validated and confirm that the findings on the association between Taurine predicted levels using NIR wavelengths and observed “Woody Breast”. The findings supportive of the systems, methodologies, processes and compositions show that for each unit decrease in Taurine, a 14% decrease in the incidence of severe Woody Breast is observed (adjusted R-squared: .69), which confirms the efficacy and utility of the technology as disclosed and claimed herein

Further evaluations focusing on Taurine, showed a strong association between Taurine levels and woody breast severity (r=.74); even considering the subjective nature of the calls for WB, the highest taurine levels were only encountered for the most severely affected breast muscles. Although not with as strong association with Woody Breast, other amino acids showed negative correlation with Taurine levels, and in certain implementations are further used in a predictive model.

### NIR Data Pre-Processing and Prediction Models

Near-infrared (NIR) spectral data pre-processing is an integral part of chemometrics modeling. There is no substitute for optimal data collection, but, after proper data collection, pre-processing of spectral data is a key step before chemometric modeling. The following corrections have been evaluated.
Standard Normal Variate (SNV): Normalizes each row of an input matrix by subtracting each row by its mean and dividing it by its standard deviation. Follows a standard normal distribution, having a mean (average) of 0 and a standard deviation of 1, allowing for comparison and analysis of data from different normal distributions by transforming them into a standardized form. It is obtained by transforming a normal random variable (X) with mean (μ) and standard deviation (σ) into a Z-score using the formula: Z=(X−μ)/σ. This transformation essentially “standardizes” the data, allowing you to compare it to the standard normal distribution and make inferences about probabilities and other statistical properties.
Multiplicative Scatter Correction (MSC): Multiplicative scatter correction method which attempts to remove physical light scatter by accounting for additive and multiplicative effects, which is a pre-processing technique used in spectroscopy to minimize the effects of light scattering and path length variations on spectral data. It is utilized to correct spectra by fitting a linear model between each spectrum and a reference spectrum, often the average of all spectra in the dataset. This correction helps to reduce multiplicative deviations caused by scattering, making the spectra more comparable and improving the accuracy of subsequent analysis.
Detrending Spectral Correction (DTR): Normalizes each row of an input matrix by applying a SNV transformation followed by fitting a second order linear model and returning the fitted residuals. Spectral corrections are needed to correct flux estimates for low and high frequency losses due to the instrument setup, intrinsic sampling limits of the instruments, and some data processing choices.
First Derivative with Sawitzky-Goley filtering (SG1): Savitzky-Golay smoothing and first derivative.
Second Derivative with Savitzky-Goley filtering (SG2), which is a method for smoothing data, particularly useful for noisy signals, by fitting a polynomial to a window of data points and using the polynomial to estimate the central data point's smoothed value. It excels at preserving signal characteristics like peak heights and widths while reducing noise. It's often used in spectroscopy and other signal processing applications: Savitzky-Golay smoothing and second derivative, which are utilized to smooth data and calculate its derivatives, including the second derivative, simultaneously. This is useful for analyzing noisy data, particularly in spectroscopy and signal processing, by reducing noise while preserving the signal's overall shape and characteristics. The filter works by fitting a polynomial to a moving window of data points and replacing the center point with the polynomial's value or derivative.
Gap-Segment Derivatives (GSD): Gap-Segment and first derivatives, which is utilized as a spectral pre-processing technique used to enhance spectral data, particularly in applications like spectroscopy and chemometrics. It involves calculating the derivative of a signal by averaging values over segments separated by a defined gap, allowing for tunable smoothing and differentiation. This method is an alternative to Savitzky-Golay (SG) smoothing derivatives and can be particularly useful for optimizing signal-to-noise ratios and resolving spectral features.
lsmeans (LS-means, least squared means), in statistics is also known as estimated marginal means, are predicted means from a statistical model, specifically for unbalanced designs. They represent the estimated marginal means of a variable over a hypothetical balanced population, essentially adjusting for the effects of other variables in the model. LS-means are commonly used in ANOVA and other linear models to compare means across different groups, particularly when those groups have unequal sample sizes. In the context of the LSMEANS statement in SAS, it computes and compares the LS-means of fixed effects. The LSMEANS statement allows users to specify which effects they want to estimate LS-means for and to perform comparisons (e.g., p-differences) between them, according to SAS documentation. The lsmeans R package (and its successor emmeans) provides functions to calculate LS-means, contrasts, and other comparisons.
tukey also known as Tukey's HSD or Tukey's range test, is a statistical test used for multiple comparisons after an ANOVA (Analysis of Variance, is a statistical method used to compare the means of two or more groups to determine if there's a statistically significant difference between them. It essentially analyzes the variance within and between groups to assess whether the observed differences are likely due to actual population differences or just random chance). It helps determine which specific group means differ significantly from each other when an ANOVA test has already indicated a significant difference among the group means. Tukey's test is often used in conjunction with ANOVA to perform pairwise comparisons of all group means. An ANOVA is performed, and if a significant difference is found, Tukey's test is applied. The test calculates a value (w) based on the error term from the ANOVA and the number of groups being compared. If the absolute difference between any two group means is greater than the calculated value, those two means are considered significantly different.

In NIR data, the observations show a high degree of multicollinearity. In addition, most datasets have more predictors than observations. However, the data contain 185 samples (observations) and 125 NIR (predictors). Hence, in addition to methods that rely on dimensionality reduction (PLS) a Linear Model could also be used. The following regression/classification methods have been applied.
Linear Regression (LM), which is a statistical method used to model the relationship between a dependent variable and one or more independent variables by fitting a linear equation to the observed data. It aims to find the best-fitting straight line (or hyperplane in higher dimensions) that represents the relationship between the variables. This line can then be used to predict the value of the dependent variable for new or unseen values of the independent variable(s).
Partial Least Square (PLS), which is a statistical method used to analyze data with many predictor variables, especially when those variables are highly correlated (multicollinear). It's a versatile technique that combines features of both principal component analysis and multiple regression. PLS creates new, uncorrelated variables called components, which are linear combinations of the original predictors, and uses these components to model the relationship with the response variable(s). PLS extracts a set of components (latent variables) that capture the maximum covariance between the predictor variables (X) and the response variables (Y). Once the components are extracted, PLS performs a regression analysis to predict the response variables using these components as predictors. The process of extracting components and performing regression is often repeated iteratively until a satisfactory model is achieved.
Support Vector Machine (SVM), which is a supervised machine learning algorithm used for classification and regression tasks. SVMs work by finding the optimal hyperplane that separates data points into different classes or predicts a continuous outcome. The input data is preprocessed and potentially transformed using kernel functions to handle complex relationships. SVM algorithms search for the hyperplane that maximizes the margin between classes, ensuring a robust separation of data points. Once the hyperplane is found, it's used to classify new, unseen data points or predict continuous values based on their position relative to the hyperplane.
Random Forest (RF), which is an ensemble learning method that constructs multiple decision trees during training and outputs the class selected by most trees for classification or the average prediction for regression. The algorithm creates multiple subsets of the training data by random sampling with replacement (bootstrap sampling). For each subset, a decision tree is built, but at each node split, only a random subset of features is considered. When making a prediction, each tree in the forest provides a prediction, and the final prediction is determined by: Classification-Taking the majority vote among all trees. Regression-Averaging the predictions of all trees. Random forests can also provide insights into which features are most important for making predictions.

Estimates of correlation between original variable and its prediction were performed using leave-one-out cross validation, which is a model evaluation technique where each data point in the dataset is used exactly once as a test set, while the remaining data points are used for training. This process is repeated for every data point, resulting in n separate training and testing sets (where n is the number of data points). The final evaluation score is then typically calculated by averaging the results from each iteration. For each data point in the dataset, a model is trained on all other data points. The trained model is then tested on the single, held-out data point. The process is repeated for every data point, resulting in n different test results. The final performance metric (e.g., accuracy, error rate) is calculated by averaging the results from all n iterations.

TABLE 3
Predictive ability of Taurine by method

| Variable | Method | LM | PLS | SVM | RF |
|---|---|---|---|---|---|
| Taurine | NIR | 0.705 | 0.705 | 0.355 | 0.566 |
| | MSC | 0.602 | 0.602 | 0.397 | 0.579 |
| | DTR | 0.604 | 0.604 | 0.368 | 0.610 |
| | SG1 | 0.698 | 0.698 | 0.519 | 0.659 |
| | SG2 | 0.678 | 0.678 | 0.559 | 0.667 |
| | GSD | 0.708 | 0.708 | 0.453 | 0.636 |

Prediction has been further improved from .7 to .73 using feature selection through Markov Blanket discovery (which is a technique used in Bayesian networks and causal inference to identify the smallest set of variables that renders a target variable independent of all other variables in the network. It's a crucial component in learning Bayesian network structures, feature selection for classification, and causal discovery. Algorithms for MB discovery aim to efficiently and accurately identify these crucial sets of variables) using Bayesian networks (is a probabilistic graphical model that represents the probabilistic relationships between a set of variables using a directed acyclic graph (DAG). It's a powerful tool for reasoning under uncertainty, combining prior knowledge with data to model complex systems and make predictions) using score-based algorithm (Hill Climbing-score-based algorithm is a method used to learn the structure of a model, such as a Bayesian network, by evaluating the “goodness of fit” of different candidate structures using a scoring function. It then searches for the structure that maximizes this score. Essentially, it's a search problem where the algorithm explores the space of possible structures, guided by the chosen scoring function). A scoring function is defined by choosing a metric to evaluate the “quality” of a network structure. A search algorithm (e.g., greedy search, simulated annealing, genetic algorithms) is utilized to explore the space of directed acyclic graph (DAGs). For each candidate structure, calculate its score using the defined scoring function. The algorithm aims to find the structure that achieves the highest score, indicating the best fit to the data.

TABLE 4
Markov blanket for Taurine includes (81 selected):

c(“NIR.908”, “NIR.914”, “NIR.920”, “NIR.926”, “NIR.939”, “NIR.945”,
“NIR.951”, “NIR.963”, “NIR.970”, “NIR.976”, “NIR.982”, “NIR.1007”,
“NIR.1013”, “NIR.1019”, “NIR.1031”, “NIR.1038”, “NIR.1050”, “NIR.1056”,
“NIR.1062”, “NIR.1069”, “NIR.1081”, “NIR.1087”, “NIR.1093”, “NIR.1106”,
“NIR.1137”, “NIR.1149”, “NIR.1155”, “NIR.1162”, “NIR.1168”, “NIR.1174”,
“NIR.1193”, “NIR.1199”, “NIR.1217”, “NIR.1236”, “NIR.1242”, “NIR.1248”,
“NIR.1261”, “NIR.1267”, “NIR.1279”, “NIR.1292”, “NIR.1298”, “NIR.1378”,
“NIR.1391”, “NIR.1397”, “NIR.1403”, “NIR.1409”, “NIR.1416”, “NIR.1428”,
“NIR.1434”, “NIR.1453”, “NIR.1471”, “NIR.1477”, “NIR.1484”, “NIR.1490”,
“NIR.1496”, “NIR.1502”, “NIR.1508”, “NIR.1521”, “NIR.1527”, “NIR.1533”,
“NIR.1539”, “NIR.1546”, “NIR.1552”, “NIR.1558”, “NIR.1564”, “NIR.1570”,
“NIR.1577”, “NIR.1583”, “NIR.1589”, “NIR.1595”, “NIR.1601”, “NIR.1608”,
“NIR.1614”, “NIR.1620”, “NIR.1626”, “NIR.1632”, “NIR.1639”, “NIR.1645”,
“NIR.1651”, “NIR.1657”, “NIR.1663”)

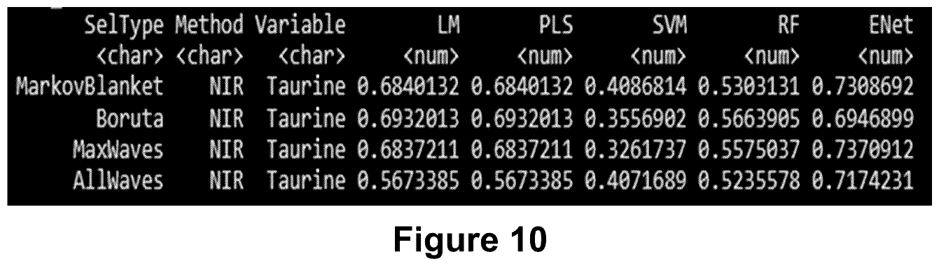

Referring to FIG. 10, provided is R Output: Predictive ability based on feature selection type and model (LM: Linear model; PLS: Partial Least Squares; SVM: Support Vector Machine; RF: Random Forest; Enet: Elastic Net). Figure shows predictive ability based on Model and variable selection type while using leave-one-out validation approach. Method Enet (or Elastic Nets) showed highest accuracy values (from .69 to .73 depending on the variable selection method).

Partial Least Square (PLS), which is a statistical method used to analyze data with many predictor variables, especially when those variables are highly correlated (multicollinear). It's a versatile technique that combines features of both principal component analysis and multiple regression. PLS creates new, uncorrelated variables called components, which are linear combinations of the original predictors, and uses these components to model the relationship with the response variable(s). PLS extracts a set of components (latent variables) that capture the maximum covariance between the predictor variables (X) and the response variables (Y). Once the components are extracted, PLS performs a regression analysis to predict the response variables using these components as predictors. The process of extracting components and performing regression is often repeated iteratively until a satisfactory model is achieved.

Support Vector Machine (SVM), which is a supervised machine learning algorithm used for classification and regression tasks. SVMs work by finding the optimal hyperplane that separates data points into different classes or predicts a continuous outcome. The input data is preprocessed and potentially transformed using kernel functions to handle complex relationships. SVM algorithms search for the hyperplane that maximizes the margin between classes, ensuring a robust separation of data points. Once the hyperplane is found, it's used to classify new, unseen data points or predict continuous values based on their position relative to the hyperplane.

Random Forest (RF), which is an ensemble learning method that constructs multiple decision trees during training and outputs the class selected by most trees for classification or the average prediction for regression. The algorithm creates multiple subsets of the training data by random sampling with replacement (bootstrap sampling). For each subset, a decision tree is built, but at each node split, only a random subset of features is considered. When making a prediction, each tree in the forest provides a prediction, and the final prediction is determined by: Classification-Taking the majority vote among all trees. Regression-Averaging the predictions of all trees. Random forests can also provide insights into which features are most important for making predictions.

Additional Internal Trials with Pooled Data (3.14 AA, FOSS)

Higher Taurine levels IS SIGNIFICANTLY associated with higher resolution pH (higher Ph also associates to white stripes, histidine, and fat), higher rigidity of the left side of the breast, higher white stripes scores, higher fat (from foss) and lower histidine levels. Hydroxyproline was only significantly associated with histidine.

Additional trial data, apart from the original 190 birds, was collected on 555 birds. The information confirms statistical significance association between Woody Breast and Taurine level prediction based on NIR technology and also Taurine obtained from tissue chemistry.

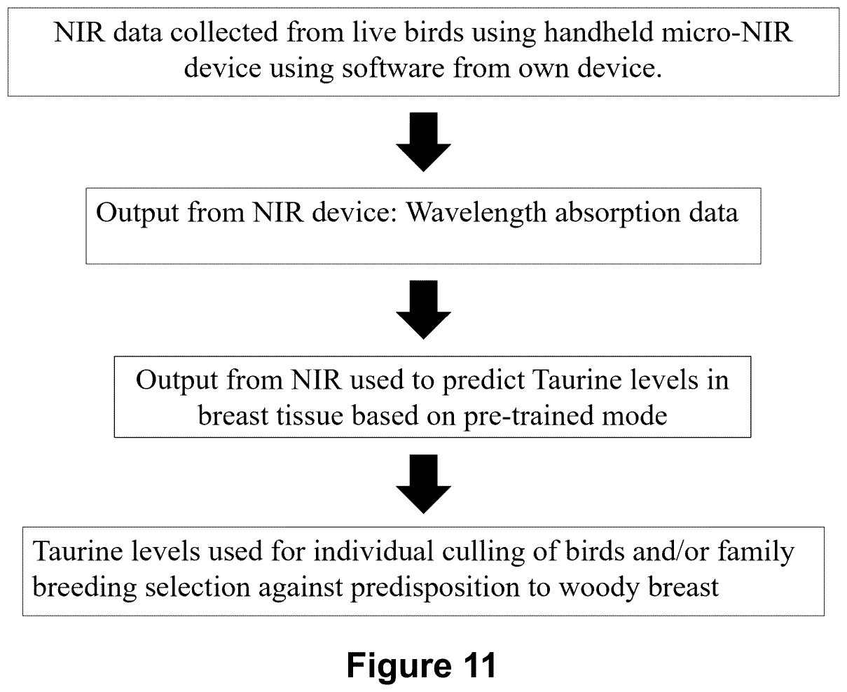

Referring to FIG. 11, provided is a Flow Diagram for processing using NIR detection. NIR data is collected from live birds using a micro-NIR scanning device having a computer programmed with software for capturing absorption and reflection data across various wavelengths. The computer is further programmed to output from the device wavelength absorption to a secondary computing device programmed with a data and software model that predicts Taurine levels in the breast tissue of the scanned bird based on a pre-trained mode. The predicted Taurine levels are used for individual culling of birds and/or family breeding selection against a predisposition to Woody Breast.

### Process Description

In certain implementations, NIR data is collected on the top left side of the breast of live birds using handheld micro-NIR device using software from own device, wherein the output from the NIR device is wavelength absorption data. After which wavelength absorption data is Sent to cloud storage. In certain implementations, output from NIR is used as input in trained model containing a weight factors for each wavelength and creates a prediction of taurine level. In certain implementations, Taurine levels are used for individual culling of birds and/or family breeding selection against predisposition to woody breast.

Algorithms for Taurine prediction utilization wavelength absorption output from NIR: Lasso, Ridge Regression and the Elastic Net

Response variable (Tau) Y∈ [symbol] and the predictors (NIR: Waive Length) X∈ [symbol] , and with approximation of the regression function by a linear model E(Y|X=x)=β₀+x^{T}β. With N observation pairs (xᵢ, yᵢ), where xᵢⱼ are standardized such that

$$\sum i = 1 NXij = 0 \sum i = 1 Nxij = 0$$

$$and$$

$$\sum i = 1 NX 2 ij = 1 \sum i = 1 Nxij 2 = 1$$

for j=l, . . . , p. The elastic net algorithm solves the following problem.

$${min}_{( β 0 , β ) ∈ {R}_{p + 1}} [ 12 N {\sum }_{i - 1 N} {( {y}_{i} - {β}_{0} - {x}_{Ti} β )}_{2} + λ {P}_{α} ( β ) ] min β0 , β ∈ ℝp + 1 12 N \sum i - 1 Nyi - β 0 - xiT β2 + λ P αβ$$

$$where$$

$${P}_{α} ( β ) = ( 1 - α ) 12 {‖ β ‖}_{2 {ℓ}_{2}} + α {‖ β ‖}_{{ℓ}_{1}} = {\sum }_{i - 1 , p} [ 12 ( 1 - α ) {β}_{2 j + α} \vert β \vert ] P αβ = 1 - α12βℓ22 + αβℓ1 = {\sum }_{j} = 1 p 121 - αβ j 2 + αβ$$

is the elastic-net penalty (Zou and Hastie, 2005). Pα is a compromise between the ridge-regression penalty (α=0) and the lasso penalty (α=1). This penalty is useful in the p>>N situation, or any situation where there are many correlated predictor variables.

Ridge regression is known to shrink the coefficients of correlated predictors towards each other, allowing them to borrow strength from each other. In the extreme case of k identical predictors, they each get identical coefficients with 1/kwh the size that any single one would get if fit alone. From a Bayesian point of view, the ridge penalty is ideal if there are many predictors, and all have non-zero coefficients (drawn from a Gaussian distribution).

Lasso, on the other hand, is somewhat indifferent to very correlated predictors, and will tend to pick one and ignore the rest. The Lasso penalty corresponds to a Laplace prior, which expects many coefficients to be close to zero, and a small subset to be larger and nonzero.

The elastic net with α=1−ε for some small ε>0 performs much like the lasso, but removes any degeneracies and wild behavior caused by extreme correlations. More generally, the entire family Pα creates a useful compromise between ridge and lasso. As a increases from 0 to 1, for a given λ the sparsity of the solution to (1) (i.e. the number of coefficients equal to zero) increases monotonically from 0 to the sparsity of the Lasso solution.

The predictions were performed using the glmnet R package (Friedman et al. 2010), where α varied depending on the type of prediction model. For ridge regression (Hoerl and Kennard 1970) we set α=0, for Lasso (Tibshirani 1996) we set α=1, and for Elastic Net, where we want to use a sparse model but are worried about correlations between features, we set α=0.5. The final value of λ was chosen via cross-validation: we select the coefficients corresponding to the λ value giving smallest cross-validated error as the final model.

### Prediction

Estimates of correlation between the original variable (Tau) and its prediction was performed using leave-one-out cross validation. Four models were tested: Linear Regression, Ridge Regression, Lasso, and Elastic Net.

A total of 189 samples and 125 wavelengths were used in the analysis. The predictive ability of each model was 0.64 (Linear Regression), 0.67 (Ridge Regression), 0.72 (Lasso), and 0.74 (Elastic Net). Hence, the Elastic Net model (higher predictive ability) was chosen to be used for prediction of Tau.

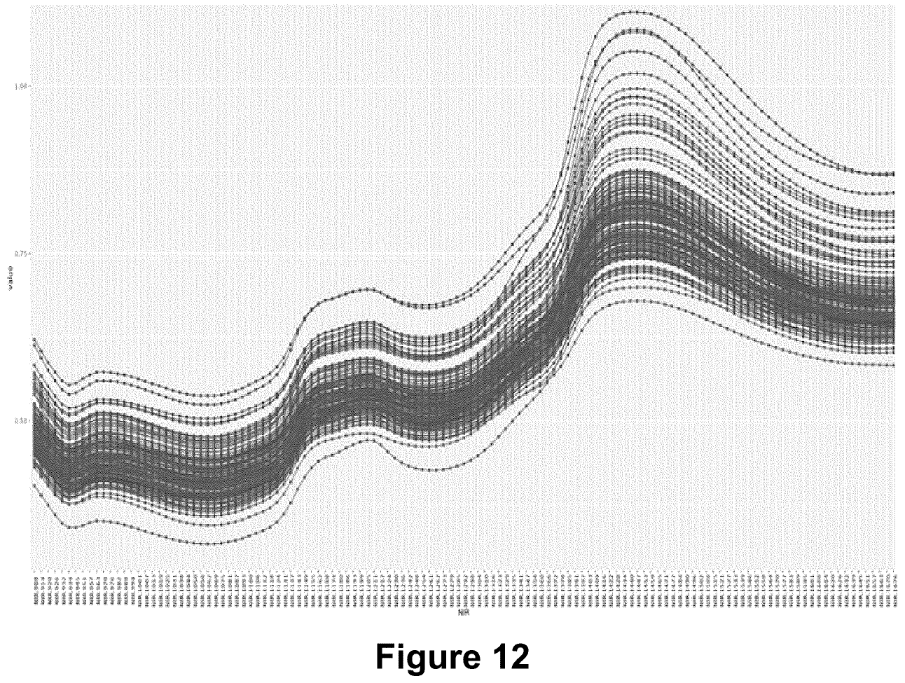

FIG. 12—Example NIR output for 146 chickens; absorption of each NIR wavelength by chicken.

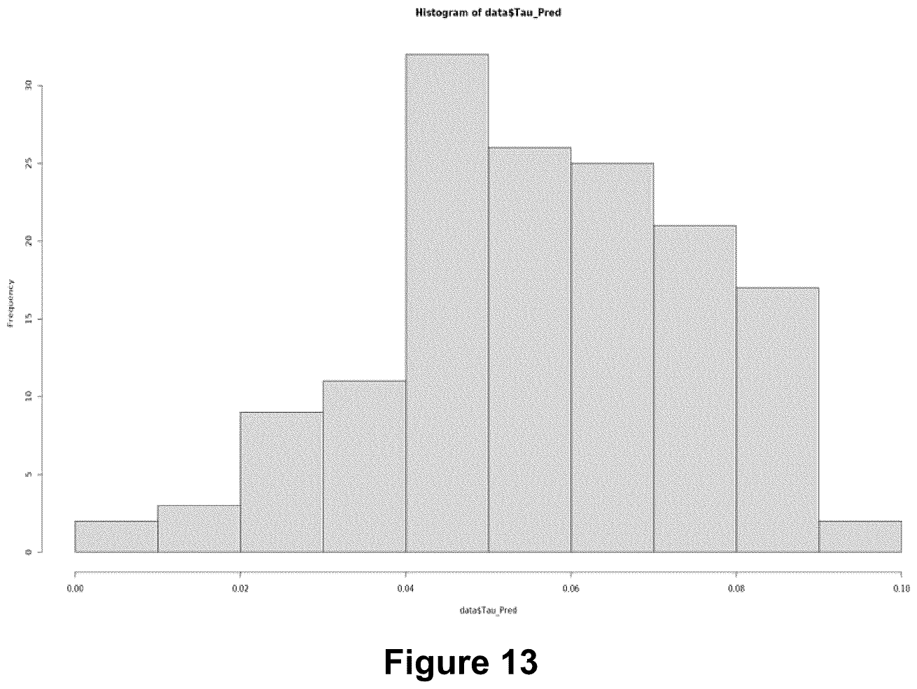

FIG. 13—Taurine predicted values for 146 birds as described herein.

Results—Blindly predicted Taurine levels were statistically significantly associated with woody breast, e.g. Table 3.

TABLE 5
ANOVA statistical significance analysis

| Tau~ | | | | F | | |
|---|---|---|---|---|---|---|
| UADxWB | Df | Sum Sq | Mean Sq | Value | Pr(>F) | Signif |
| Woody breast | 3 | 0.00756498 | 0.002522 | 8.1387 | 4.87E−05 | *** |
| Residuals | 142 | 0.04399666 | 0.00031 | | | |

### Additional Datasets

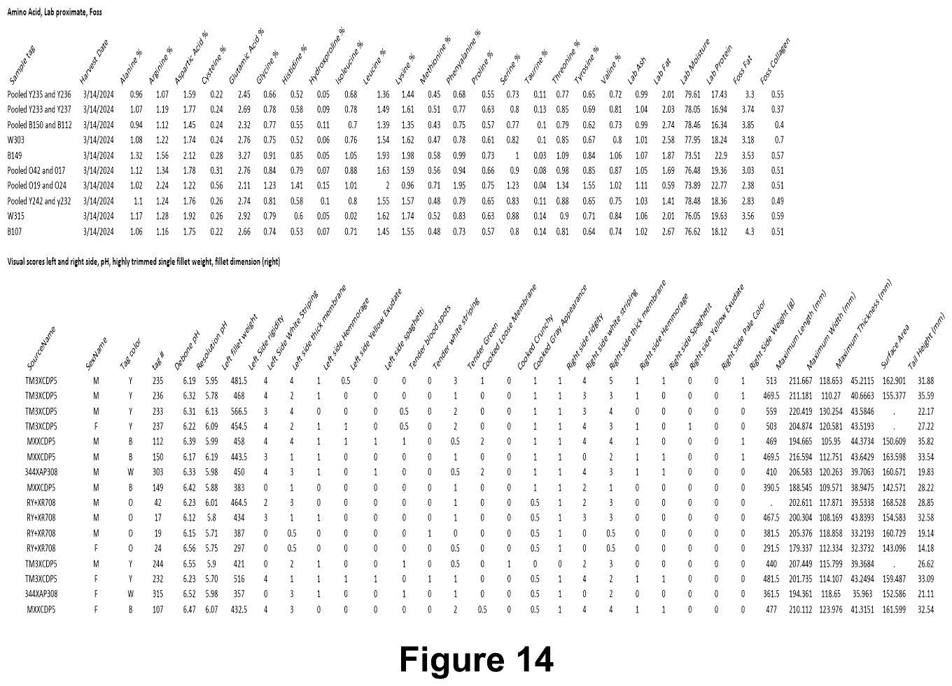

FIG. 14—Data 3.14

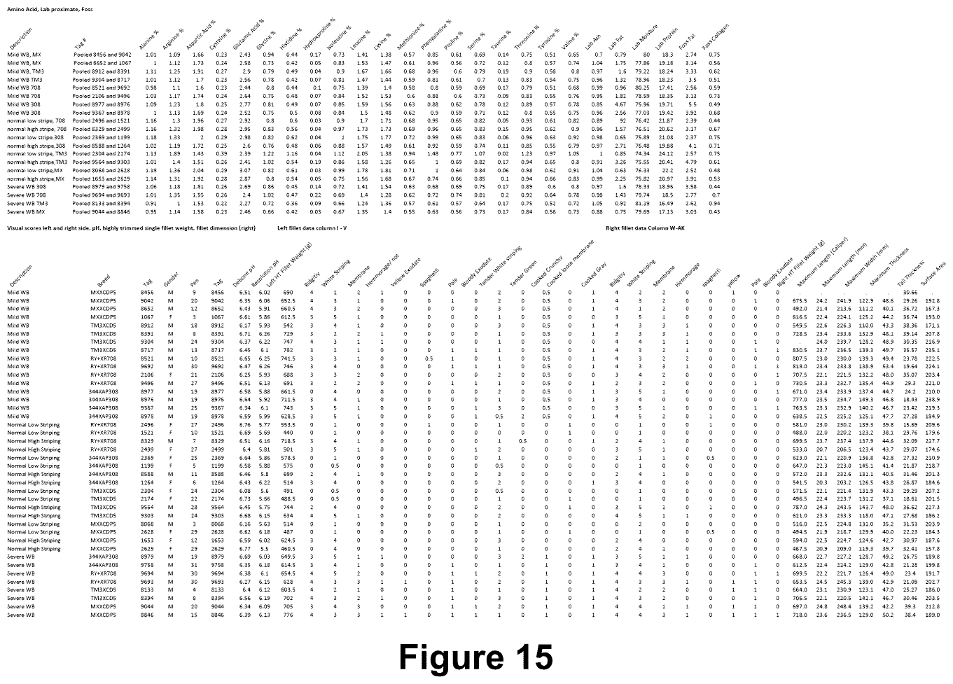

FIG. 15—Data 3.28

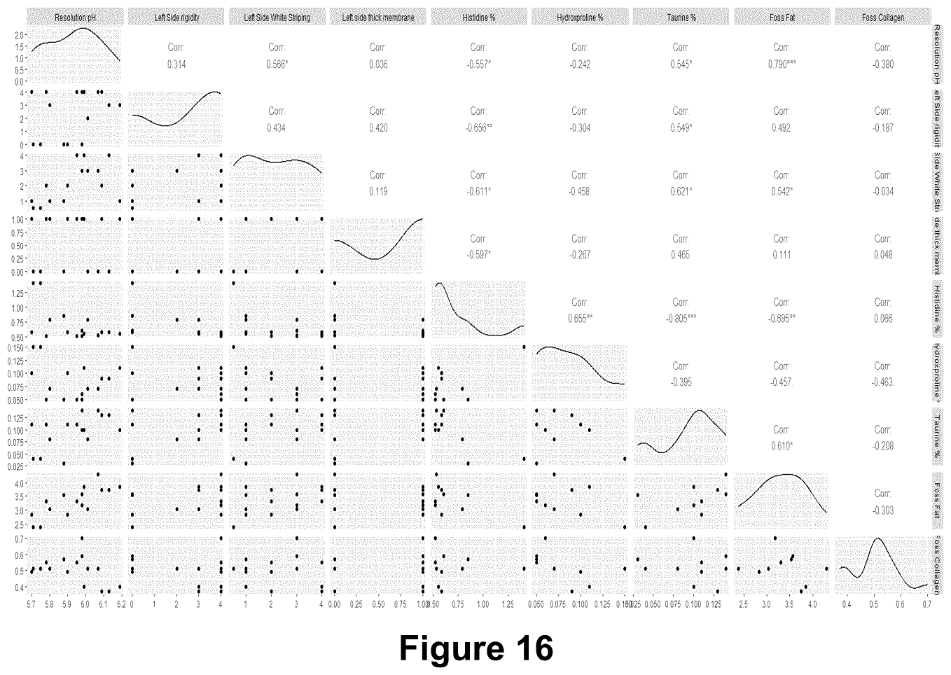

FIG. 16—Pooled 3.14 and 3.28 data results. Higher Taurine levels significantly associated with higher resolution pH (higher pH also associates to white stripes, histidine, and fat), higher rigidity of the left side of the breast, higher white stripes scores, higher fat (from foss) and lower histidine levels. Hydroxyproline was only significantly associated to histidine.

### Follow on Subsequence Lab Analysis for Pooled Data.

Data on 20 samples tested for AA profile and protein/fat/moisture content. A very large outlier for LabFat was present (value=92), probably due to typing mistake. One data point for histidine and lab ash.

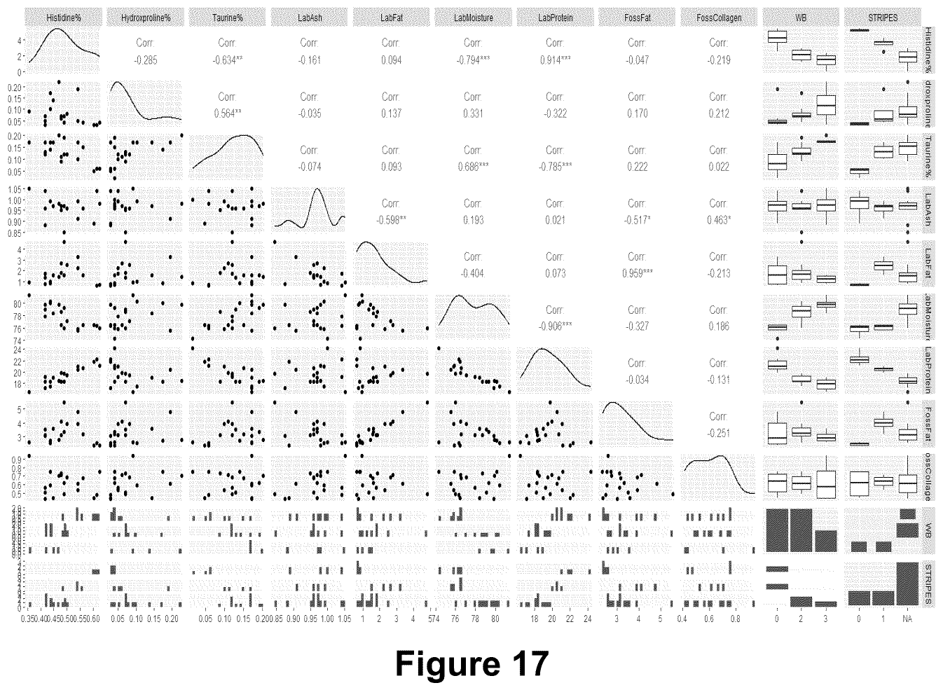

Results—The level of woody breast is associated with higher levels of taurine/lab moisture, lower levels of histidine/Lab Protein. High Taurine levels can also be found on high WS scored breasts, even if WB is not present, summarized as FIG. 17. Same as observed in other trials, which validates our previous findings. Interestingly, high Taurine levels can also be found on high WS scored breasts, even if WB is not present. Samples were pooled, so it is hard to separate those two traits. Again, the level of woody breast (0=normal, 2=Mild and 3=Extreme) is associated with higher levels of taurine/lab moisture, lower levels of histidine/Lab Protein.

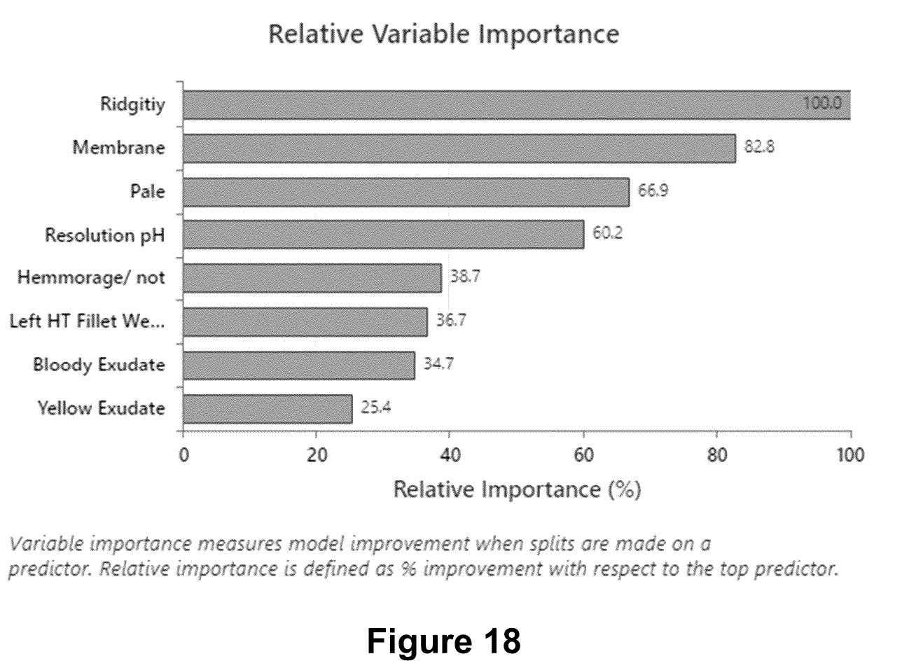

Further, “cooked crunchy” which is a woody breast eating defect has rigidity as the most important variable for prediction, followed by membrane, pale and resolution pH (left side of fillet), as shown in FIG. 18.

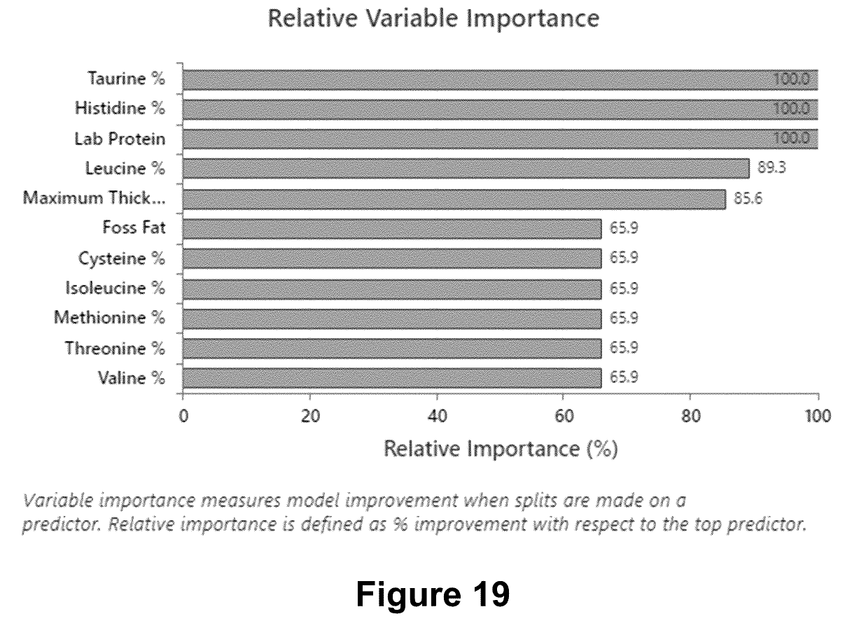

When taking pooled data which is then modeled for rigidity: Taurine, Histidine and Protein % are the most important variables, as shown in FIG. 19.

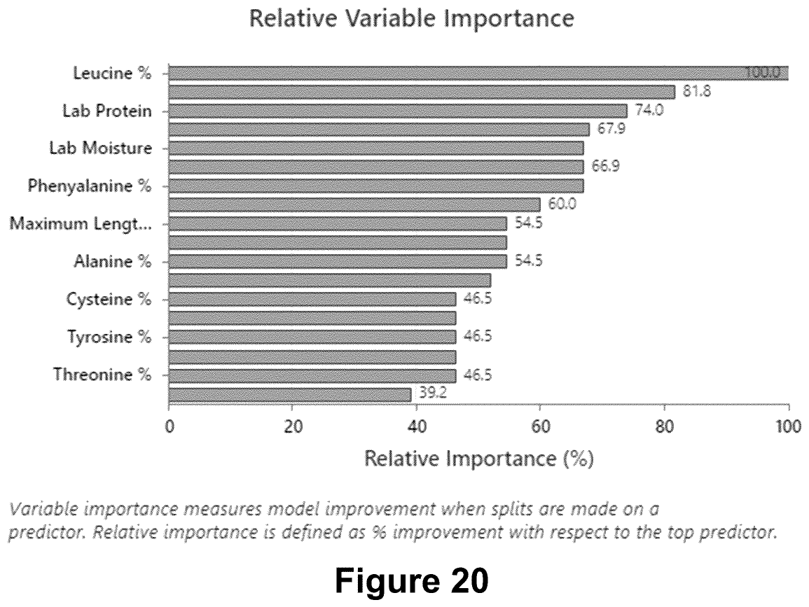

When taking pooled data which is then modeled for pale: leucine rises as most important variable, as shown in FIG. 20.

One implementation of the technology as disclosed and claimed herein is a method for detection of a breast muscle disorder including one or more myopathies in a live poultry subject including scanning an upper left side breast muscle area of a live poultry subject with a micro-NIR-Infrared scanning device of an NIR spectrometer emitting light in a near-infrared spectrum. The process further includes detecting with a light sensor of the scanning device, a plurality of reflected wavelengths of light; capturing and generating with software processing on a computing device having a processor integral with the scanner device data representative of a plurality of absorbed and the plurality of reflected wavelengths of light based on the light sensor detection; and analyzing the data representative of the absorbed wavelengths of light and determining a presence and a concentration of various properties within the scanned breast muscle. For one implementation, the process further includes outputting the data representative of the presence and concentration of various properties from the scanning device to a Taurine scoring model on a secondary computing device for absorbed wavelengths between approximately 908 nm and 1676 nm and determining a Taurine level of the breast muscle with the scoring model; and outputting the Taurine level from the scoring model to the genetic/genomic evaluation model thereby determining a breeding value. One implementation further includes identifying the poultry subject with lower or higher predisposition to one or more breast muscle myopathies based on the Taurine level.

The process for detection of a breast muscle disorder as disclosed and claimed herein can be utilized to detect one or more of Woody Breast, White Striping, and Spaghetti Meat. For one implementation, the process for detection of a breast muscle disorder as disclosed and claimed herein includes selecting poultry subjects in a breeding program with lower predisposition to Woody Breast based on the Taurine level and the determined breeding value. One implementation of the process includes removing poultry subjects from a poultry production line for alternative processing with a predisposition to Woody Breast based on the Taurine level, and where the Taurine level is approximately 0.15 to 0.55.

One implementation of the process as disclosed and claimed herein for detection in a live chicken subject includes, obtaining spectroscopic absorption data via non-invasive near infrared spectroscopy, at one or more wavelengths, of the anatomical breast area of the live chicken subject; processing and analyzing the spectroscopic absorption data to determine the presence of one or more diseases, one or more characteristics, one or more compounds, or combination thereof; and processing the subject in one or more predetermined manners based on the presence of one or more diseases, one or more characteristics, one or more compounds, or combination thereof.

One implementation for detection in a subject as disclosed and claimed herein, includes, obtaining spectroscopic absorption data via spectroscopy, at one or more wavelengths, of one or more anatomical areas of the subject, relevant to (a) one or more diseases, (b) one or more characteristics, (c) one or more compounds, or (d) a combination thereof; processing and analyzing the spectroscopic absorption data to determine whether one or more thresholds is reached in the subject; and wherein if the one or more thresholds is reached, processing the subject in one or more predetermined manners based on the one or more thresholds, and wherein the subject is an individual, a group, a population, or a combination thereof, and wherein the spectroscopy is non-invasive and the subject is alive during the spectroscopy.

For one implementation of the technology the process of detection is that of one or more diseases including (a) wooden breast, (b) white striping, (c) spaghetti meat or (d) a combination thereof, wherein the one or more compounds, includes one or more amino acids. For one implementation the one or more amino acids, includes taurine. For one implementation the one or more anatomical areas of the subject is (a) the upper side of breast, (b) the pectoris major muscle, or (c) a combination thereof, wherein the spectroscopic absorption data comprises data from gamma radiation, x-ray radiation, ultraviolet radiation, visible light, infrared radiation, microwave radiation, radio waves, or a combination thereof, and wherein the spectroscopic absorption data comprises data from infrared radiation.

Other aspects, objects and advantages of the present technology as disclosed herein can be obtained from a study of the drawings, the disclosure and the appended claims.

While the present invention is susceptible of embodiments in many different forms, there is shown in the drawings and will herein be described in detail preferred implementations of the present invention with the understanding that the present disclosure is to be considered as an exemplification of the principles of the present invention and is not intended to limit the broad aspect of the present invention to the embodiments illustrated.

Many modifications and variations of the present invention are possible in light of the above teachings. It is, therefore, to be understood within the scope of the appended claims the invention may be protected otherwise than as specifically described.

I/We claim:

1. A method for detection of a breast muscle disorder including one or more myopathies in a live poultry subject comprising:
scanning an upper portion of a breast muscle area of a live poultry subject with a micro-NIR-Infrared scanning device of an NIR spectrometer emitting light in a near-infrared spectrum;
detecting with a light sensor of the scanning device, a plurality of reflected NIR wavelengths of light;
capturing and generating with software processing on a computing device, having a processor integral with the scanner device, data representative of a plurality of absorbed and the plurality of reflected wavelengths of light based on the light sensor detection;
analyzing the data representative of the absorbed wavelengths of light and determining a presence and a concentration of various properties within the scanned breast muscle;
outputting data representative of the presence and the concentration of various properties from the scanning device to a Taurine scoring model on a secondary computing device for absorbed wavelengths between approximately 908 nm and 1676 nm and determining a Taurine level of the breast muscle with the scoring model;
outputting the Taurine level from the scoring model to the genetic/genomic evaluation model thereby determining a breeding value; and
identifying the poultry subject with lower or higher predisposition to one or more breast muscle myopathies based on the Taurine level.

2. The method for detection of a breast muscle disorder as recited in claim 1, where scanning the upper portion of the breast muscle area is scanning above the upper left portion of the breast muscle area where the one or more myopathies is one or more of Woody Breast, White Striping, and Spaghetti Meat.

3. The method for detection of a breast muscle disorder as recited in claim 2, comprising:
selecting poultry subjects in a breeding program with lower predisposition to Woody Breast based on the Taurine level and the determined breeding value.

4. The method for detection of a breast muscle disorder as recited in claim 2, comprising:
removing poultry subjects from a poultry production line for alternative processing with a predisposition to Woody Breast based on the Taurine level.

5. The method for detection of a breast muscle disorder as recited in claim 4, where the Taurine level is approximately 0.15 to 0.55.

6. The method for detection of a breast muscle disorder as recited in claim 3, where the Taurine level is approximately 0.15 to 0.55.

7. A method for detection of a muscle myopathy in a live chicken subject comprising:
obtaining spectroscopic absorption data via a non-invasive near infrared spectroscopy, at one or more wavelengths, of the anatomical breast area of a feathered live chicken subject;
processing and analyzing the spectroscopic absorption data to determine the presence of one or more myopathies, one or more characteristics, one or more compounds, or combination thereof; and
processing the subject in one or more predetermined manners based on the presence of one or more diseases, one or more characteristics, one or more compounds, or combination thereof based on a correlation between the analyzed spectroscopic absorption data and one or more amino acids.

8. A method for detection in a live poultry subject comprising:
obtaining spectroscopic absorption data via spectroscopy, at one or more wavelengths, of one or more anatomical areas of the subject, relevant to (a) one or more diseases, (b) one or more characteristics, (c) one or more compounds, or (d) a combination thereof;
processing and analyzing the spectroscopic absorption data to determine whether one or more thresholds within the absorption data is reached in the subject; and wherein if the one or more thresholds is reached, processing the subject in one or more predetermined manners based on the one or more thresholds where the one or more threshold has been correlated to (a) one or more diseases, (b) one or more characteristics, (c) one or more compounds, or (d) a combination thereof, and
wherein the subject is an individual, a group, a population, or a combination thereof, and wherein the spectroscopy is non-invasive and the subject is alive during the spectroscopy.

9. The method of claim 8 wherein the one or more diseases is (a) wooden breast, (b) white striping, (c) spaghetti meat or (d) a combination thereof.

10. The method of claim 8, wherein the one or more compounds comprises, one or more amino acids.

11. The method of claim 10, wherein the one or more amino acids comprises Taurine.

12. The method of claim 8, wherein the one or more anatomical areas of the subject is (a) the upper side of breast, (b) the pectoris major muscle, or (c) a combination thereof.

13. The method of claim 8, wherein the spectroscopic absorption data comprises data from gamma radiation, x-ray radiation, ultraviolet radiation, visible light, infrared radiation, microwave radiation, radio waves, or a combination thereof.

14. The method of claim 8, wherein the spectroscopic absorption data comprises data from infrared radiation.

15. The method of claim 8, wherein the spectroscopic absorption data is data from near infrared radiation.

16. The method claim 8, wherein the one or more wavelengths, in nanometers, is 908, 914, 920, 926, 939, 945, 951, 963, 970, 976, 982, 1007, 1013, 1019, 1031, 1038, 1050, 1056, 1062, 1069, 1081, 1087, 1093, 1106, 1137, 1149, 1155, 1162, 1168, 1174, 1193, 1199, 1217, 1236, 1242, 1248, 1261, 1267, 1279, 1292, 1298, 1378, 1391, 1397, 1403, 1409, 1416, 1428, 1434, 1453, 1471, 1477, 1484, 1490, 1496, 1502, 1508, 1521, 1527, 1533, 1539, 1546, 1552, 1558, 1564, 1570, 1577, 1583, 1589, 1595, 1601, 1608, 1614, 1620, 1626, 1632, 1639, 1645, 1651, 1657, 1663, or a combination of thereof.

17. The method of claim 8, wherein the one or more characteristics is quality, tenderness, aesthetic appearance, or a combination thereof.

18. The method of claim 8, wherein the subject is a chicken.

19. The method of claim 8, wherein the subject is a broiler chicken.

20. The method of claim 8, wherein the one or more predetermined manners is (a) destruction of the subject, (b) elimination of the subject from one or more genetic breeding programs, (c) use of the subject for alternative processing, or (d) a combination thereof.
